(19) United States
(12) Patent Application Publication
(10) Pub. No.: US 2020/0054601 A1
(43) Pub. Date: Feb. 20, 2020

(54) NOVEL METHODS

(71) Applicant: Ethismos Research, Inc., Cambridge, MA (US)

(72) Inventor: Anthony MCKINNEY, Newton Center, MA (US)

(21) Appl. No.: 16/362,525

(22) Filed: Mar. 22, 2019

Related U.S. Application Data
(60) Provisional application No. 62/646,904, filed on Mar. 22, 2018.

(51) Int. Cl. A61K 31/40

(52) U.S. Cl. CPC A61K 31/40

(57) ABSTRACT

Provided are methods of treatment for nicotine dependency and nicotine withdrawal effects (e.g., a method of smoking cessation) in a human in need thereof comprising administering to the human an effective amount of (1R,5S)-1-(3,4-dichlorophenyl)-3-azabicyclo[3.1.0]hexane, in free or pharmaceutically acceptable salt form.

This application claims priority to U.S. Provisional Application No. 62/646,904 filed Mar. 22, 2018, the contents of which are hereby incorporated by reference.

### BACKGROUND

Over 1 billion people smoke worldwide. Currently, tobacco is the single greatest preventable cause of death in the world. According to the Centers for Disease Control and Prevention, in 2015, nearly 68% of adult U.S. cigarette smokers wanted to stop smoking and more than 55% of adult U.S. cigarette smokers had made a quit attempt in the past year. Though approximately 30% of U.S. smokers utilize medications and/or behavioral counseling to quit only 7% of quit attempts are successful. It is recognized that currently available smoking cessation medications and behavioral interventions can reduce craving but significant residual withdrawal effects, peaking immediately after the quit attempt and remaining elevated for days and sometimes for weeks and months later, are a major reason for relapse. Studies have shown that current medications are capable of reducing craving (one withdrawal symptom) to baseline but negative affect, the depression-like symptoms following cessation of any drug of abuse, remain elevated even with optimal current therapies. Increase in negative affect and loss of positive affect or the positive feelings that accompany smoking in addition to the other sequalae of smoking cessation (e.g., weight gain) lead to relapse.

Currently approved medications for smoking cessation include nicotine replacement therapy (NRT), bupropion, and varenicline. However, these medications only provide 6-month abstinence rates of 17.6% for NRT, 19.1% for bupropion, and 27.6% for varenicline compared to 10.6% for placebo. Importantly, one year abstinence rates for NRT, bupropion, and varenicline are 10%, 15%, and 23%, respectively.

New medications are needed for those who attempt to quit smoking with full courses of standard pharmacological therapy yet are refractory or relapse.

### BRIEF SUMMARY

Provided is a method of treatment for nicotine dependency and nicotine withdrawal effects (e.g., a method of smoking cessation) in a human in need thereof, wherein the method comprises administering to the human an effective amount of (1R,5S)-1-(3,4-dichlorophenyl)-3-azabicyclo[3.1.0]hexane, in free or pharmaceutically acceptable salt form, wherein the human has previously been refractory to at least one (e.g., one or two or more) adequate prior course of approved treatment for nicotine dependency and nicotine withdrawal effects (e.g., wherein the human has previously been refractory to at least one adequate prior course of approved treatment for smoking cessation (e.g., a nicotine replacement therapy)) or wherein the human has relapsed after at least one adequate prior course of successful approved treatment for nicotine dependency and nicotine withdrawal effects (e.g., wherein the human has relapsed after at least one adequate prior course of successful approved treatment for smoking cessation (e.g., a nicotine replacement therapy)) (e.g., wherein the human has previously been refractory) (e.g., wherein the human has relapsed).

Further provided is a method of treatment for nicotine dependency and nicotine withdrawal effects (e.g., a method of smoking cessation) in a human in need thereof, wherein the method comprises:
a. determining and/or learning if the human is a poor, a slow, an intermediate, a normal, or a fast nicotine metabolizer (e.g., by measuring nicotine metabolite ratio),
b. determining an effective amount of (1R,5S)-1-(3,4-dichlorophenyl)-3-azabicyclo[3.1.0]hexane, in free or pharmaceutically acceptable salt form, based on whether the human is a poor, a slow, an intermediate, a normal, or a fast nicotine metabolizer, and
c. administering the effective amount of (1R,5S)-1-(3,4-dichlorophenyl)-3-azabicyclo[3.1.0]hexane, in free or pharmaceutically acceptable salt form, to the human.
   For instance, if the human is a poor or slow nicotine metabolizer, then the effective amount of (1R,5S)-1-(3,4-dichlorophenyl)-3-azabicyclo[3.1.0]hexane, in free or pharmaceutically acceptable salt form, is 25 mg to 75 mg per day, for example 25 mg or 50 mg per day. Or, if the human is an intermediate, a normal, or a fast nicotine metabolizer, then the effective amount of (1R,5S)-1-(3,4-dichlorophenyl)-3-azabicyclo[3.1.0]hexane, in free or pharmaceutically acceptable salt form, is 75 mg to 200 mg per day, for example, 100 mg to 200 mg per day.

Further provided is a method of treatment for nicotine dependency and nicotine withdrawal effects (e.g., a method of smoking cessation) in a human in need thereof, wherein the method comprises administering to the human an effective amount of (1R,5S)-1-(3,4-dichlorophenyl)-3-azabicyclo[3.1.0]hexane, in free or pharmaceutically acceptable salt form, and an effective amount of a nicotine replacement therapy (e.g., an e-cigarette or “vape” or a nicotine patch, gum, lozenge, inhaler, or nasal spray or oral nicotine (e.g., tablet or capsule)), wherein the human is an intermediate, a normal, or a fast nicotine metabolizer (e.g., an intermediate nicotine metabolizer or a normal nicotine metabolizer or a fast nicotine metabolizer).

Further provided is a method of treatment for nicotine dependency and nicotine withdrawal effects (e.g., a method of smoking cessation) in a human in need thereof, wherein the method consists essentially of administering to the human an effective amount of (1R,5S)-1-(3,4-dichlorophenyl)-3-azabicyclo[3.1.0]hexane, in free or pharmaceutically acceptable salt form, wherein the human is a poor or slow nicotine metabolizer.

Further provided is a method of inhibiting the metabolism of nicotine to cotinine in a human in need thereof, wherein the method comprises administering to the human an effective amount of (1R,5S)-1-(3,4-dichlorophenyl)-3-azabicyclo[3.1.0]hexane, in free or pharmaceutically acceptable salt form, wherein the human is addicted or has a dependency to nicotine. Further provided is a method of inhibiting the metabolism of cotinine to 3′-hydroxycotinine (e.g., trans-3′-hydroxycotinine (3-HC)) in a human in need thereof, wherein the method comprises administering to the human an effective amount of (1R,5S)-1-(3,4-dichlorophenyl)-3-azabicyclo[3.1.0]hexane, in free or pharmaceutically acceptable salt form, wherein the human is addicted or has a dependency to nicotine.

Further provided is a method of enhancing the effectiveness of a nicotine replacement therapy (e.g., an e-cigarette or “vape” or a nicotine patch, gum, lozenge, inhaler, or nasal spray or oral nicotine (e.g., tablet or capsule)) in a human in need thereof, wherein the method comprises administering to the human an effective amount of (1R,5S)-1-(3,4-dichlorophenyl)-3-azabicyclo[3.1.0]hexane, in free or pharmaceutically acceptable salt form, wherein the human is addicted or has a dependency to nicotine and wherein the human is an intermediate, a normal, or a fast nicotine metabolizer (e.g., an intermediate nicotine metabolizer or a normal nicotine metabolizer or a fast nicotine metabolizer).

Further provided is a method for treatment or prophylaxis of lung cancer or other cancer associated with tobacco use in a human in need thereof, wherein the method comprises administering to the human an effective amount of (1R,5S)-1-(3,4-dichlorophenyl)-3-azabicyclo[3.1.0]hexane, in free or pharmaceutically acceptable salt form.

Further provided is a method of reducing one or more withdrawal effects (symptoms) following quitting smoking in a human in need thereof, wherein the method comprises administering to the human an effective amount of (1R,5S)-1-(3,4-dichlorophenyl)-3-azabicyclo[3.1.0]hexane, in free or pharmaceutically acceptable salt form, wherein the human is addicted or has a dependency to nicotine and wherein the human is an intermediate, normal, or fast nicotine metabolizer (e.g., an intermediate nicotine metabolizer or a normal nicotine metabolizer or a fast nicotine metabolizer).

Further provided is a method of reducing one or more withdrawal effects (symptoms) following quitting smoking in a human in need thereof, wherein the method comprises:
a. determining and/or learning if the human is a poor, a slow, an intermediate, a normal, or a fast nicotine metabolizer (e.g., by measuring nicotine metabolite ratio),
b. determining an effective amount of (1R,5S)-1-(3,4-dichlorophenyl)-3-azabicyclo[3.1.0]hexane, in free or pharmaceutically acceptable salt form, based on whether the human is a poor, a slow, an intermediate, a normal, or a fast nicotine metabolizer, and
c. administering the effective amount of (1R,5S)-1-(3,4-dichlorophenyl)-3-azabicyclo[3.1.0]hexane, in free or pharmaceutically acceptable salt form, to the human.

Further provided is a method of treatment for nicotine dependency and nicotine withdrawal effects (e.g., a method of smoking cessation) in a human in need thereof, wherein the method comprises administering to the human an effective amount of (1R,5S)-1-(3,4-dichlorophenyl)-3-azabicyclo[3.1.0]hexane, in free or pharmaceutically acceptable salt form, at least 1 week before target quit smoking date, e.g., 1 week before, e.g., 2 weeks before, e.g., 3 weeks before, e.g., 4 weeks before.

Further provided is a method of preventing smoking relapse following quitting smoking in a human in need thereof, wherein the method comprises administering to the human an effective amount of (1R,5S)-1-(3,4-dichlorophenyl)-3-azabicyclo[3.1.0]hexane, in free or pharmaceutically acceptable salt form, wherein the human is an intermediate, a normal, or a fast nicotine metabolizer.

Further provided is a method of preventing smoking relapse following quitting smoking in a human in need thereof, wherein the method comprises:
a. determining and/or learning if the human is a poor, a slow, an intermediate, a normal, or a fast nicotine metabolizer (e.g., by measuring nicotine metabolite ratio),
b. determining an effective amount of (1R,5S)-1-(3,4-dichlorophenyl)-3-azabicyclo[3.1.0]hexane, in free or pharmaceutically acceptable salt form, based on whether the human is a poor, a slow, an intermediate, a normal, or a fast nicotine metabolizer, and
c. administering the effective amount of (1R,5S)-1-(3,4-dichlorophenyl)-3-azabicyclo[3.1.0]hexane, in free or pharmaceutically acceptable salt form, to the human.

Further provided is a method of increasing nicotine bioavailability in a human dependent on tobacco (e.g., human smoker) in need thereof, wherein the method comprises administering to the human an effective amount of (1R,5S)-1-(3,4-dichlorophenyl)-3-azabicyclo[3.1.0]hexane, in free or pharmaceutically acceptable salt form.

Further provided is a method of enabling oral nicotine (e.g., tablet or capsule) for use (e.g., for chronic use) as a nicotine replacement therapy in a human in need thereof, wherein the method comprises administering an effective amount of (1R,5S)-1-(3,4-dichlorophenyl)-3-azabicyclo[3.1.0]hexane, in free or pharmaceutically acceptable salt form, in combination with oral nicotine to the human.

Further provided is a method of treatment of nicotine dependence and nicotine withdrawal effects (e.g., a method of smoking cessation) and smoking relapse prevention in a human in need thereof, wherein the method comprises administering an effective amount of (1R,5S)-1-(3,4-dichlorophenyl)-3-azabicyclo[3.1.0]hexane, in free or pharmaceutically acceptable salt form, in combination with oral nicotine (e.g., tablet or capsule) to the human.

Further areas of applicability of the present disclosure will become apparent from the detailed description provided hereinafter. It should be understood that the detailed description and specific examples, while indicating preferred embodiments of this disclosure, are intended for purposes of illustration only and are not intended to limit the scope of this disclosure.

### BRIEF DESCRIPTION OF THE DRAWINGS

The present disclosure will become more fully understood from the detailed description and the accompanying drawings, wherein:

FIG. 1 shows inhibition of CYP2A6 coumarin 7-hydroxylation in human liver microsomes by amitifadine: IC₅₀ determination.

FIG. 2 shows dosing of amitifadine, nicotine replacement therapy, and placebo in study evaluating efficacy of amitifadine in terms of promoting smoking abstinence and reducing smoking withdrawal symptoms.

### DETAILED DESCRIPTION

The following description of preferred embodiments is merely exemplary in nature and is in no way intended to limit this disclosure, its application, or uses.

As used throughout, ranges are used as shorthand for describing each and every value that is within the range. Any value within the range can be selected as the terminus of the range. In the event of a conflict in a definition in the present disclosure and that of a cited reference, the present disclosure controls.

(+)-1-(3,4-dichlorophenyl)-3-azabicyclo[3.1.0]hexane, also known as (1R,5S)-1-(3,4-dichlorophenyl)-3-azabicyclo[3.1.0]hexane, also known amitifadine, is shown as Formula I below.

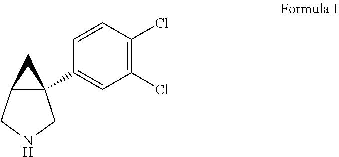 [SMILES: Clc1ccc([C@]23CNC[C@H]2C3)cc1Cl]

“(+)-1-(3,4-dichlorophenyl)-3-azabicyclo[3.1.0]hexane,” “(1R,5S)-1-(3,4-dichlorophenyl)-3-azabicyclo[3.1.0]hexane,” and “amitifadine” are used interchangeably herein. (1R,5S)-1-(3,4-dichlorophenyl)-3-azabicyclo[3.1.0]hexane is an unbalanced triple reuptake inhibitor with the greatest potency towards inhibiting serotonin reuptake (5-HT), half as much towards inhibiting norepinephrine reuptake (NE), and one eighth as much towards inhibiting dopamine reuptake (DA). (1R,5S)-1-(3,4-dichlorophenyl)-3-azabicyclo[3.1.0]hexane hydrochloride is reported to inhibit the reuptake of [³H]serotonin, [³H]norepinephrine, and [³H]dopamine in human embryonic kidney (HEK) 293 cells expressing the corresponding human recombinant transporters at IC₅₀ values of 12, 23, and 96 nm, respectively.

(1R,5S)-1-(3,4-dichlorophenyl)-3-azabicyclo[3.1.0]hexane may be synthesized as described in U.S. Pat. Nos. 6,372,919, 7,098,229, and 9,527,813, each of which are hereby incorporated by reference in their entirety.

Efficacy data on dual and triple reuptake inhibitors in major depressive disorder (MDD) have been variable. For example, SEP-225289, a norepinephrine and dopamine reuptake inhibitor (now called dasotraline) was initially in development for MDD. However, in a phase 2 trial versus venlafaxine or placebo, SEP-225289 failed to differentiate versus the comparators and was discontinued for MDD. GSK-372475 (also known as NS-2359), a balanced triple, failed in two large phase 2b trials in MDD. Liafensine (BMS-820836), a serotonin-preferring triple, was evaluated in two large phase 2b trials in treatment resistant depression (TRD) randomizing TRD patients to liafensine or active comparators and found to be no different from full-dose escitalopram (an SSRI) or duloxetine (an SNRI).

With respect to smoking cessation, venlafaxine, a serotonin-norepinephrine reuptake inhibitor, has not been shown to help. For duloxetine, a serotonin-norepinephrine reuptake inhibitor, it is reported that smoking affects its bioavailability.

The atypical antidepressant bupropion, a norepinephrine-dopamine reuptake inhibitor, is approved for smoking cessation. Bupropion or its metabolites may also function as antagonists at nicotinic acetylcholine receptors. It has been reported that the tricyclic antidepressant nortriptyline, which preferentially inhibits reuptake of norepinephrine and more weakly inhibits reuptake of serotonin, increases long-term smoking cessation when used as the sole pharmacotherapy. However, nortriptyline is not labeled for smoking cessation and has the potential for serious side-effects. In addition, nortriptyline in combination with nicotine patch therapy has not shown evidence of an additional benefit from nortriptyline.

For selective serotonin reuptake inhibitors (SSRIs), one open-label trial found that fluoxetine in combination with a transdermal nicotine patch for smoking cessation in people with elevated symptoms of depression had significantly higher rates of abstinence than standard treatment with fluoxetine at the 6-month assessment, but that difference was reduced at the 12-month assessment. In addition, a systematic review that took into account that open-label trial still concluded SSRIs have not been shown to help smoking cessation.

Withdrawal effects following smoking cessation are closely correlated with smoking relapse. Negative affect following smoking cessation is frequently associated with lapse (episodes of smoking after a period of abstinence). Lapses nearly always result in relapse. One study found that smoking relapse is associated with (in order): post-quit sleep disturbance, a history of failed quit attempt in the previous 3 months, a history of depression and anxiety in the previous 3 months, exposure to smoking cues, the baseline level of nicotine dependence, availability of smoking cessation aids (e.g., medications), and post-quit weight related factors. Anhedonia has been recently evaluated and found to meet criteria for a clinically significant and independent tobacco withdrawal symptom. But, the failure of SSRIs in helping smoking cessation shows that even though a medication may be effective in treating depression does not necessarily equate to success in helping smoking cessation.

Cigarette smoking produces a rapid distribution of nicotine through the bloodstream and nicotine crosses the blood-brain barrier. Studies have shown that smokers will try to maintain their plasma nicotine concentration within a narrow range by modulating their smoking behavior. This may include altering the number of cigarettes smoked per day or how they smoke a cigarette (e.g., depth of inhalation).

Cytochrome P450 2A6 (CYP2A6) is a monooxygenase enzyme that metabolizes xenobiotic compounds and activates toxins. Human CYP2A6 enzyme activity may be determined by measuring metabolism of a CYP2A6 substrate (e.g., nicotine).

The CYP2A6 gene is highly polymorphic, with over 40 different CYP2A6 alleles described, with additional subgroups. CYP2A6 genotypes are often classified into predicted phenotype groups, describing the effect on enzyme activity, for example “poor” metabolizer (no active CYP2A6 alleles, homozygous for inactive alleles, or one inactive and one decreased activity CYP2A6 alleles), “slow” metabolizer (one inactive or two decreased activity CYP2A6 alleles), “intermediate” metabolizer (heterozygous with one decreased activity CYP2A6 allele and one active CYP2A6 allele), “normal”/“extensive” (two active CYP2A6 alleles), or “ultrarapid”/“fast” (greater than two active CYP2A6 alleles).

Cotinine (COT) is the major metabolite of nicotine in humans. Trans-3′-hydroxycotinine (3-HC) is the major metabolite of cotinine. In vitro and in vivo studies show that CYP2A6 is primarily responsible for the oxidation of nicotine and cotinine.

Genetic variability in CYP2A6 has been associated with a variety of smoking behavior phenotypes. Reduced or null activity CYP2A6 alleles (e.g., CYP2A6*9, CYP2A6*12, CYP2A6*2, or CYP2A6*4) are more prevalent in non-smokers than smokers. Smokers with reduced or null activity smoke fewer cigarettes and tend to be less dependent on nicotine than smokers with normal activity alleles. With respect to smoking initiation, adolescents with normal activity alleles may progress to nicotine dependence more quickly than slower metabolizers.

Studies have reported associations of CYP2A6 activity with smoking cessation outcome and response to treatment. Compared to faster nicotine metabolizers (higher nicotine metabolite ratio, greater CYP2A6 activity), slower nicotine metabolizers exhibit higher quit rates following 8 weeks of treatment with nicotine patch.

Smokers with high activity CYP2A6 report more serious nicotine withdrawal symptoms during smoking cessation. Smokers with high activity CYP2A6 smoke the first cigarette earlier upon arising in the morning than those with low activity, indicating more severe nicotine dependence than those with low activity. This refers to the Time to First Cigarette (TTFC), a validated proxy for nicotine dependence.

Research indicates that withdrawal symptoms interfere with smoking cessation. Faster metabolizers of nicotine may experience more intense withdrawal symptoms due to a more rapid decline in blood and brain nicotine concentrations after smoking a cigarette. In adolescent light smokers, faster metabolizers report greater withdrawal symptoms after 24 h and describe themselves as more highly addicted than slow metabolizers.

One method to determine variation in nicotine metabolism is to determine the nicotine metabolite ratio (NMR), which is the ratio of the concentration (e.g., in ng/ml) of nicotine metabolites trans-3′-hydroxycotinine (3HC or 3-HCOT) to cotinine (COT) (3HC/COT). Measurement of NMR is a validated approach for determining in vivo CYP2A6 enzyme activity among smokers and can be measured in different biological matrices, including blood, plasma, saliva, and urine. Null or reduced activity CYP2A6 alleles have lower NMRs.

Tobacco smoke contains a number of tobacco-specific procarcinogen nitrosamines, e.g., N-nitrosodiethylamine, 4-(methylnitrosamino)-1-(3-pyridyl)-1-butanone, and N′-nitrosonornicotine, that CYP2A6 can activate via α-hydroxylation. Therefore, individuals who have low CYP2A6 activity may also be less efficient at bioactivating tobacco smoke procarcinogens to carcinogens, whereas those with high CYP2A6 activity may be more efficient.

It has been reported that having the CYP2A6*4 allele results in a significant reduction in risk for lung cancer. The decreased risk could be due to the gene's impact on amount smoked (decreasing exposure to carcinogens) and/or on decreased activation of procarcinogens. It has also been reported that after adjusting for age, sex, race/ethnicity, body mass index, smoking duration, and urinary creatinine levels, both total nicotine equivalents (TNE) (higher smoking) and higher CYP2A6 activity are associated with an increase in lung cancer risk. The association for CYP2A6 activity remains even after adjusting for cigarettes per day (CPD) and TNE.

A CYP2A6 slow nicotine metabolizer phenotype may be mimicked by use of a CYP2A6 inhibitor.

Amitifadine has been shown to reduce nicotine self-administration in an animal model. However, it has not been previously reported that amitifadine is a CYP2A6 inhibitor. Yet, as shown by Example 1, amitifadine is a potent CYP2A6 inhibitor. As a potent inhibitor of CYP2A6, the primary metabolic pathway of nicotine, amitifadine slows inactivation of nicotine in the brain thereby prolonging and elevating nicotine levels in the brain. This pharmacological “phenocopying” mimics CYP2A6 slow metabolizers that show greater responsiveness to nicotine replacement therapy, less withdrawal effects, and higher success in smoking cessation.

Without being bound by theory, CYP2A6 inhibition by amitifadine contributes to high efficacy of amitifadine/nicotine replacement therapy (NRT) combination treatment wherein amitifadine is administered before the target quit-smoking date both because (1) inhibition of CYP2A6 before the target quit-smoking date attenuates reinforcement associated with post-cigarette nicotine peaks, leading to a reduction in addiction severity and cigarette consumption and (2) inhibition of CYP2A6, and the resulting longer half-life of nicotine, will yield higher nicotine brain and plasma levels from the NRT.

Based on the finding that amitifadine is a potent inhibitor of CYP2A6, and may thus induce a reduction in smoking rate prior to the quit date, pre-quit treatment with amitifadine may be particularly important in maximizing the chances for a successful clinical outcome.

Regarding nicotine withdrawal effects, smoking cessation treatments may help with craving, but do not have all the pharmacology to address withdrawal symptoms associated with smoking cessation. For example, bupropion modulates norepinephrine and dopamine but has a comparatively modest 0.27 effect size. Nortriptyline is a potent tricyclic antidepressant with considerable side effects though with norepinephrine and serotonin activity but no dopamine. Amitifadine modulates all of serotonin, norepinephrine, and dopamine and has a 0.6 effect size in depression and has shown good tolerability when given by mouth twice daily for up to 12 weeks. The serotonin, norepinephrine, and dopamine reuptake pharmacology of amitifadine allow it to address reasons smokers relapse: post-quit negative affect/depression symptoms as well as lack of positive affect (anhedonia). Amitifadine also addresses impulsivity and craving. Finally, amitifadine may attenuate quit-related weight gain.

The dual facets of amitifadine as a CYP2A6 inhibitor and triple reuptake inhibitor endows amitifadine with a unique pharmacological profile compared to other treatments for smoking cessation. Without being bound by theory, it is the unique pharmacological profile of amitifadine that allows it to be successful in smokers who have previously not responded or failed smoking cessation treatment, for instance, people with high activity CYP2A6, and in smokers who have relapsed after an initial period of abstinence.

The maximum tolerable dose of oral nicotine is around 4 mg, because larger doses may cause gastric irritation. Upon first pass through the liver, the majority of nicotine is metabolized to cotinine, reducing bioavailable nicotine. The high first-pass metabolism, coupled with the intestinal disturbances caused by high nicotine doses, limits use of oral nicotine in nicotine replacement therapy because oral delivery cannot provide sufficient nicotine to substitute for smoked nicotine. However, administering, oral nicotine in combination with amitifadine, a CYP2A6 inhibitor, may increase bioavailable nicotine, thereby making oral nicotine a viable nicotine replacement therapy.

As used herein, “(+)-1-(3,4-dichlorophenyl)-3-azabicyclo[3.1.0]hexane,” “(1R,5S)-1-(3,4-dichlorophenyl)-3-azabicyclo[3.1.0]hexane,” and “amitifadine” embraces the compound in any form, for example, free or pharmaceutically acceptable salt form, e.g., as a pharmaceutically acceptable acid addition salt. Pharmaceutically acceptable salts are known in the art and include salts that are physiologically acceptable at the dosage amount and form to be administered, for example, hydrochloride salts.

As used herein, “(+)-1-(3,4-dichlorophenyl)-3-azabicyclo[3.1.0]hexane,” “(1R,5S)-1-(3,4-dichlorophenyl)-3-azabicyclo[3.1.0]hexane,” and “amitifadine” is also to be understood as embracing the compound in crystalline and amorphous form including, for example, polymorphs, solvates (including hydrates), unsolvated polymorphs (including anhydrates), conformational polymorphs, and amorphous forms of the compounds, as well as mixtures thereof. “Crystalline form” and “polymorph” may be used interchangeably herein, and are meant to include all crystalline forms of (1R,5S)-1-(3,4-dichlorophenyl)-3-azabicyclo[3.1.0]hexane, in free or pharmaceutically acceptable salt form, including, for example, polymorphs, solvates (including hydrates), unsolvated polymorphs (including anhydrates), and conformational polymorphs, as well as mixtures thereof, unless a particular crystalline form is referred to.

(1R,5S)-1-(3,4-dichlorophenyl)-3-azabicyclo[3.1.0]hexane exists in at least three polymorphic forms, labeled polymorphs A, B, and C, as disclosed in U.S. Pat. Nos. 8,765,801, 9,139,521, and 9,770,436, each of which are hereby incorporated by reference in their entirety.

Crystalline and amorphous forms of (1R,5S)-1-(3,4-dichlorophenyl)-3-azabicyclo[3.1.0]hexane, in free or pharmaceutically acceptable salt form, may be used in any combination or in forms that are substantially free of one or more of the other crystalline forms or free of the amorphous form.

As used herein, “substantially free of other polymorphic forms” means that the crystalline material contains no more than 5% w/w of any other crystalline form, e.g., no more than 2% w/w of any other crystalline form, e.g., no more than 1% w/w of any other crystalline form.

(1R,5S)-1-(3,4-dichlorophenyl)-3-azabicyclo[3.1.0]hexane may in some cases also exist in prodrug form. Prodrugs are considered to be any covalently bonded carriers that release the active parent drug in vivo.

As used herein, “effective amount” refers to an amount effective, when administered to a human, to provide a therapeutic benefit such as reducing nicotine dependency or aiding in the cessation or lessening of tobacco use and/or amelioration of nicotine withdrawal effects (symptoms). The specific dose of substance(s) (e.g., (1R,5S)-1-(3,4-dichlorophenyl)-3-azabicyclo[3.1.0]hexane, in free or pharmaceutically acceptable salt form, and/or nicotine replacement therapy) administered to obtain a therapeutic benefit will, of course, be determined by the particular circumstances surrounding the case, including, for example, the route of administration and individual being treated, in particular, whether the individual is a poor, a slow, an intermediate, a normal, or a fast nicotine metabolizer. “Effective amount” can vary depending on whether the individual is a poor, a slow, an intermediate, a normal, or a fast nicotine metabolizer and whether (1R,5S)-1-(3,4-dichlorophenyl)-3-azabicyclo[3.1.0]hexane, in free or pharmaceutically acceptable salt form, is administered alone or in combination with another therapy, for example, a nicotine replacement therapy (e.g., a nicotine patch). “Effective amount” may vary depending on whether the individual is a poor, a slow, an intermediate, a normal, or a fast nicotine metabolizer and whether (1R,5S)-1-(3,4-dichlorophenyl)-3-azabicyclo[3.1.0]hexane, in free or pharmaceutically acceptable salt form, is administered alone or in combination with another therapy, for example, a nicotine replacement therapy of varying form, e.g., immediate release forms such as an electronic cigarette or “vape,” a sublingual spray, a nasal spray, or a pulmonary inhaler, intermediate release formulations such as lozenges or gums, or a slower release but longer acting form, e.g., a nicotine patch. “Effective amount” of a nicotine replacement therapy (e.g., a nicotine patch) can also vary depending on whether the individual is a poor, a slow, an intermediate, a normal, or a fast nicotine metabolizer and the dose of (1R,5S)-1-(3,4-dichlorophenyl)-3-azabicyclo[3.1.0]hexane, in free or pharmaceutically acceptable salt form. For instance, a slow nicotine metabolizer may be administered a lower dose of (1R,5S)-1-(3,4-dichlorophenyl)-3-azabicyclo[3.1.0]hexane, in free or pharmaceutically acceptable salt form, plus a lower dose of nicotine replacement therapy compared to an intermediate, a normal, or a fast nicotine metabolizer. In any of the methods disclosed herein, the effective amount of (1R,5S)-1-(3,4-dichlorophenyl)-3-azabicyclo[3.1.0]hexane, in free or pharmaceutically acceptable form, and the effective amount of nicotine replacement therapy may be adjusted on the basis of whether the human is a poor, a slow, an intermediate, a normal, or a fast nicotine metabolizer (e.g., determined by CYP2A6 genotype or phenotype and/or NMR) and/or whether (1R,5S)-1-(3,4-dichlorophenyl)-3-azabicyclo[3.1.0]hexane, in free or pharmaceutically acceptable form, and the nicotine replacement therapy are being administered alone or in combination with each other or another therapy and/or the form of the nicotine replacement therapy (e.g., electronic cigarette or “vape,” sublingual spray, nasal spray, pulmonary inhaler, lozenge, gum, nicotine patch).

Where two active agents are administered (e.g., (1R,5S)-1-(3,4-dichlorophenyl)-3-azabicyclo[3.1.0]hexane, in free or pharmaceutically acceptable salt form, and a nicotine replacement therapy), the effective amount of each agent may be below the amount needed for activity of each as a monotherapy. Accordingly, a subthreshold amount (i.e., an amount below the level necessary for efficacy as monotherapy) may be an effective amount. Indeed, an advantage of administering different agents with different mechanisms of action and different side effect profiles may be to reduce the dosage and side effects of either or both agents, as well as to enhance or potentiate their activity as monotherapy.

As used herein, “nicotine replacement therapy” (NRT) refers to a treatment for taking nicotine by means other than tobacco. Nicotine replacement therapy comes in a variety of forms, for example, a patch, a gum, a lozenge, an inhaler, and a nasal spray or for example, an electronic cigarette (e-cigarette) or “vape.” In an e-cigarette, the nicotine may be dissolved in a liquid that is vaporized and taken into the lungs without burning tobacco. Nicotine replacement therapy also includes oral nicotine delivered by tablet or capsule.

As used herein, “combination” means the treatments are co-administered within the same therapeutic regimen. The individual treatments may be dosed separately (e.g., a human may wear a nicotine patch for 24 hours and also take (1R,5S)-1-(3,4-dichlorophenyl)-3-azabicyclo[3.1.0]hexane, in free or pharmaceutically acceptable form, orally twice daily) or together (e.g., simultaneously or within the same composition).

(1R,5S)-1-(3,4-dichlorophenyl)-3-azabicyclo[3.1.0]hexane, in free or pharmaceutically acceptable form, administered according to the methods described herein (e.g., any of Methods 1-15 or 1.1-1.162) may be expected to provide tonic-like protection against withdrawal effects, because (1R,5S)-1-(3,4-dichlorophenyl)-3-azabicyclo[3.1.0]hexane, in free or pharmaceutically acceptable form, may have sustained effects in the brain since it operates on a post-synaptic basis and may be given twice daily, not according to symptoms. This offers clinicians flexibility to augment with supplemental immediate release NRT or long-acting NRT or no NRT depending on the patient CYP profile and individual needs.

As used herein, “human has previously been refractory to at least one adequate prior course of approved treatment for nicotine dependency and nicotine withdrawal effects (e.g., wherein the human has previously been refractory to at least one adequate prior course of approved treatment for smoking cessation (e.g., a nicotine replacement therapy))” or “human has relapsed after at least one adequate prior course of successful approved treatment for nicotine dependency and nicotine withdrawal effects (e.g., wherein the human has relapsed after at least one adequate prior course of successful approved treatment for smoking cessation (e.g., a nicotine replacement therapy))” is a human who is refractory or relapsed for any reason.

In some embodiments, a refractory human may have failed to respond or failed to respond sufficiently to the adequate prior course of approved treatment. In some embodiments, a refractory human may not have responded or failed to respond sufficiently to initial treatment. In some embodiments, a refractory human may have responded to initial treatment, but relapsed during the treatment. In some embodiments, a refractory human may have been unable to continue taking the treatment because of intolerance of the treatment. Insufficient or failed responses may be determined by any means generally used, including patient self-reporting and clinical observation. An adequate prior course of approved treatment includes a complete prior course of approved treatment.

As used herein, “refractory” human includes a human who fails to become abstinent from smoking.

As used herein, “relapsed” means the human has not maintained nicotine abstinence. For instance, “relapse” includes a period of several days or more of continuous smoking after a period of abstinence or an attempt at abstinence.

As used herein, “approved treatment” means any treatment for nicotine dependency and nicotine withdrawal effects (e.g., any treatment for smoking cessation) awarded marketing approval by a national or supranational government authority that gives marketing approval for medicines, including, without limitation, the United States Food and Drug Administration, the European Medicines Agency, the Japanese Ministry of Health, Labour and Welfare, and any successors thereto.

As used herein, “administering” includes prescribing.

Nicotine metabolite ratio (NMR) is trans-3′-hydroxycotinine/cotinine (3HC/COT). Nicotine metabolite ratio may also be measured as total 3-HCOT/total COT wherein total 3-HCOT is the sum of concentrations of trans-3′-hydroxycotinine (3-HCOT) and its glucuronide conjugate and total COT is the sum of concentrations of cotinine (COT) and its glucuronide conjugate or as total 3-HCOT/COT. For measuring NMR from a urine sample, total 3-HCOT/COT may be preferred. Concentrations (e.g., in ng/ml) of 3HC, COT, total 3-HCOT, and total COT to calculate NMR may be measured by, for instance, HPLC, GC/MS, LC/MS, and immunoassay procedures as described in, for instance, Dempsey, D. et al., “Nicotine Metabolite Ratio as an Index of Cytochrome P450 2A6 Metabolic Activity,” Clinical Pharmacology & Therapeutics, 2004, 76, 64-72; Tanner, J. et al., “Nicotine Metabolite Ratio (3-Hydroxycotinine/Cotinine) in Plasma and Urine by Different Analytical Methods and Laboratories: Implications for Clinical Implementation,” Cancer Epidemiology, Biomarkers & Prevention, 2015, 24 (8), 1239-1246; St. Helen, G. et al., “Reproducibility of the Nicotine Metabolite Ratio in Cigarette Smokers,” Cancer Epidemiology, Biomarkers & Prevention, 2012, 21 (7), 1105-1114; and Malaiyandi, V. et al., “CYP2A6 Genotype, Phenotype, and the Use of Nicotine Metabolites as Biomarkers during Ad libitum Smoking,” Cancer Epidemiology, Biomarkers & Prevention, 2006, 15 (10), 1812-1819.

When discussing the activity of CYP2A6 alleles herein, the activity referenced is for metabolism of nicotine.

In the methods described herein (e.g., any of Methods 1-15 or 1.1-1.162), whether the human is a poor, a slow, an intermediate, a normal, or a fast nicotine metabolizer may be determined by measuring a ratio of nicotine metabolites (e.g., total 3-HCOT/total COT and/or 3-HCOT/COT and/or total 3-HCOT/COT). For instance, whether the human is a poor, slow, intermediate, normal, or fast nicotine metabolizer may be determined by measuring nicotine metabolite ratio (NMR). Whether the human is a poor, slow, intermediate, normal, or fast nicotine metabolizer may also determined by a CYP2A6 genotype assay and/or by a CYP2A6 phenotype assay (e.g., by measuring metabolism of nicotine) and/or by clinician assessment of the CYP2A6 genotype given the human's phenotype, for example, by considering the level of nicotine dependency using the proxy of the time to smoke the first cigarette upon arising, by considering how many cigarettes the human smokes per day, depth of inhalation of the human, and/or nicotine cessation (e.g., smoking cessation) history of the human.

Provided is a method (Method 1) of treatment for nicotine dependency and nicotine withdrawal effects (e.g., a method of smoking cessation) in a human in need thereof, wherein the method comprises administering to the human an effective amount of (1R,5S)-1-(3,4-dichlorophenyl)-3-azabicyclo[3.1.0]hexane, in free or pharmaceutically acceptable salt form, wherein the human has previously been refractory to at least one (e.g., one or two or more) adequate prior course of approved treatment for nicotine dependency and nicotine withdrawal effects (e.g., wherein the human has previously been refractory to at least one adequate prior course of approved treatment for smoking cessation (e.g., a nicotine replacement therapy)) or wherein the human has relapsed after at least one adequate prior course of successful approved treatment for nicotine dependency and nicotine withdrawal effects (e.g., wherein the human has relapsed after at least one adequate prior course of successful approved treatment for smoking cessation (e.g., a nicotine replacement therapy)) (e.g., wherein the human has previously been refractory) (e.g., wherein the human has relapsed).

Further provided is a method (Method 2) of treatment for nicotine dependency and nicotine withdrawal effects (e.g., a method of smoking cessation) in a human in need thereof, wherein the method comprises:
a. determining and/or learning if the human is a poor, a slow, an intermediate, a normal, or a fast nicotine metabolizer (e.g., by measuring nicotine metabolite ratio),
b. determining an effective amount of (1R,5S)-1-(3,4-dichlorophenyl)-3-azabicyclo[3.1.0]hexane, in free or pharmaceutically acceptable salt form, based on whether the human is a poor, a slow, an intermediate, a normal, or a fast nicotine metabolizer, and
c. administering the effective amount of (1R,5S)-1-(3,4-dichlorophenyl)-3-azabicyclo[3.1.0]hexane, in free or pharmaceutically acceptable salt form, to the human.
   For instance, if the human is a poor or slow nicotine metabolizer, then the effective amount of (1R,5S)-1-(3,4-dichlorophenyl)-3-azabicyclo[3.1.0]hexane, in free or pharmaceutically acceptable salt form, is 25 mg to 75 mg per day, for example 25 mg or 50 mg per day. Or, if the human is an intermediate, a normal, or a fast nicotine metabolizer, then the effective amount of (1R,5S)-1-(3,4-dichlorophenyl)-3-azabicyclo[3.1.0]hexane, in free or pharmaceutically acceptable salt form, is 75 mg to 200 mg per day, for example, 100 mg to 200 mg per day.

Further provided is a method (Method 3) of treatment for nicotine dependency and nicotine withdrawal effects (e.g., a method of smoking cessation) in a human in need thereof, wherein the method comprises administering to the human an effective amount of (1R,5S)-1-(3,4-dichlorophenyl)-3-azabicyclo[3.1.0]hexane, in free or pharmaceutically acceptable salt form, and an effective amount of a nicotine replacement therapy (e.g., an e-cigarette or “vape” or a nicotine patch, gum, lozenge, inhaler, or nasal spray or oral nicotine (e.g., tablet or capsule)), wherein the human is an intermediate, a normal, or a fast nicotine metabolizer (e.g., an intermediate nicotine metabolizer or a normal nicotine metabolizer or a fast nicotine metabolizer).

Further provided is a method (Method 4) of treatment for nicotine dependency and nicotine withdrawal effects (e.g., a method of smoking cessation) in a human in need thereof, wherein the method consists essentially of administering to the human an effective amount of (1R,5S)-1-(3,4-dichlorophenyl)-3-azabicyclo[3.1.0]hexane, in free or pharmaceutically acceptable salt form, wherein the human is a poor or slow nicotine metabolizer.

Further provided is a method (Method 5) of inhibiting the metabolism of nicotine to cotinine in a human in need thereof, wherein the method comprises administering to the human an effective amount of (1R,5S)-1-(3,4-dichlorophenyl)-3-azabicyclo[3.1.0]hexane, in free or pharmaceutically acceptable salt form, wherein the human is addicted or has a dependency to nicotine. Further provided is a method (Method 5a) of inhibiting the metabolism of cotinine to 3′-hydroxycotinine (e.g., trans-3′-hydroxycotinine (3-HC)) in a human in need thereof, wherein the method comprises administering to the human an effective amount of (1R,5S)-1-(3,4-dichlorophenyl)-3-azabicyclo[3.1.0]hexane, in free or pharmaceutically acceptable salt form, wherein the human is addicted or has a dependency to nicotine.

Further provided is a method (Method 6) of enhancing the effectiveness of a nicotine replacement therapy (e.g., an e-cigarette or “vape” or a nicotine patch, gum, lozenge, inhaler, or nasal spray or oral nicotine (e.g., tablet or capsule)) in a human in need thereof, wherein the method comprises administering to the human an effective amount of (1R,5S)-1-(3,4-dichlorophenyl)-3-azabicyclo[3.1.0]hexane, in free or pharmaceutically acceptable salt form, wherein the human is addicted or has a dependency to nicotine and wherein the human is an intermediate, a normal, or a fast nicotine metabolizer (e.g., an intermediate nicotine metabolizer or a normal nicotine metabolizer or a fast nicotine metabolizer).

Further provided is a method (Method 7) for treatment or prophylaxis of lung cancer or other cancer associated with tobacco use in a human in need thereof, wherein the method comprises administering to the human an effective amount of (1R,5S)-1-(3,4-dichlorophenyl)-3-azabicyclo[3.1.0]hexane, in free or pharmaceutically acceptable salt form.

Further provided is a method (Method 8) of reducing one or more withdrawal effects (symptoms) following quitting smoking in a human in need thereof, wherein the method comprises administering to the human an effective amount of (1R,5S)-1-(3,4-dichlorophenyl)-3-azabicyclo[3.1.0]hexane, in free or pharmaceutically acceptable salt form, wherein the human is addicted or has a dependency to nicotine and wherein the human is an intermediate, normal, or fast nicotine metabolizer (e.g., an intermediate nicotine metabolizer or a normal nicotine metabolizer or a fast nicotine metabolizer).

Further provided is a method (Method 9) of reducing one or more withdrawal effects (symptoms) following quitting smoking in a human in need thereof, wherein the method comprises:
a. determining and/or learning if the human is a poor, a slow, an intermediate, a normal, or a fast nicotine metabolizer (e.g., by measuring nicotine metabolite ratio),
b. determining an effective amount of (1R,5S)-1-(3,4-dichlorophenyl)-3-azabicyclo[3.1.0]hexane, in free or pharmaceutically acceptable salt form, based on whether the human is a poor, a slow, an intermediate, a normal, or a fast nicotine metabolizer, and
c. administering the effective amount of (1R,5S)-1-(3,4-dichlorophenyl)-3-azabicyclo[3.1.0]hexane, in free or pharmaceutically acceptable salt form, to the human.

Further provided is a method (Method 10) of treatment for nicotine dependency and nicotine withdrawal effects (e.g., a method of smoking cessation) in a human in need thereof, wherein the method comprises administering to the human an effective amount of (1R,5S)-1-(3,4-dichlorophenyl)-3-azabicyclo[3.1.0]hexane, in free or pharmaceutically acceptable salt form, at least 1 week before target quit smoking date, e.g., 1 week before, e.g., 2 weeks before, e.g., 3 weeks before, e.g., 4 weeks before.

Further provided is a method (Method 11) of preventing smoking relapse following quitting smoking in a human in need thereof, wherein the method comprises administering to the human an effective amount of (1R,5S)-1-(3,4-dichlorophenyl)-3-azabicyclo[3.1.0]hexane, in free or pharmaceutically acceptable salt form, wherein the human is an intermediate, a normal, or a fast nicotine metabolizer.

Further provided is a method (Method 12) of preventing smoking relapse following quitting smoking in a human in need thereof, wherein the method comprises:
a. determining and/or learning if the human is a poor, a slow, an intermediate, a normal, or a fast nicotine metabolizer (e.g., by measuring nicotine metabolite ratio),
b. determining an effective amount of (1R,5S)-1-(3,4-dichlorophenyl)-3-azabicyclo[3.1.0]hexane, in free or pharmaceutically acceptable salt form, based on whether the human is a poor, a slow, an intermediate, a normal, or a fast nicotine metabolizer, and
c. administering the effective amount of (1R,5S)-1-(3,4-dichlorophenyl)-3-azabicyclo[3.1.0]hexane, in free or pharmaceutically acceptable salt form, to the human.

Further provided is a method (Method 13) of increasing nicotine bioavailability in a human dependent on tobacco (e.g., human smoker) in need thereof, wherein the method comprises administering to the human an effective amount of (1R,5S)-1-(3,4-dichlorophenyl)-3-azabicyclo[3.1.0]hexane, in free or pharmaceutically acceptable salt form.

Further provided is a method (Method 14) of enabling oral nicotine (e.g., tablet or capsule) for use (e.g., for chronic use) as a nicotine replacement therapy in a human in need thereof, wherein the method comprises administering an effective amount of (1R,5S)-1-(3,4-dichlorophenyl)-3-azabicyclo[3.1.0]hexane, in free or pharmaceutically acceptable salt form, in combination with oral nicotine to the human.

Further provided is a method (Method 15) of treatment of nicotine dependence and nicotine withdrawal effects (e.g., a method of smoking cessation) and smoking relapse prevention in a human in need thereof, wherein the method comprises administering an effective amount of (1R,5S)-1-(3,4-dichlorophenyl)-3-azabicyclo[3.1.0]hexane, in free or pharmaceutically acceptable salt form, in combination with oral nicotine (e.g., tablet or capsule) to the human.

Further provided is any of Method 1-15 as follows:
1.1 Any of Method 1-15, wherein (1R,5S)-1-(3,4-dichlorophenyl)-3-azabicyclo[3.1.0]hexane is in pharmaceutically acceptable salt form.
1.2 Any Method 1-15 or 1.1, wherein (1R,5S)-1-(3,4-dichlorophenyl)-3-azabicyclo[3.1.0]hexane in pharmaceutically acceptable salt form is an acid addition salt.
1.3 Any of Method 1-15, 1.1, or 1.2, wherein (1R,5S)-1-(3,4-dichlorophenyl)-3-azabicyclo[3.1.0]hexane in pharmaceutically acceptable salt form is (1R,5S)-1-(3,4-dichlorophenyl)-3-azabicyclo[3.1.0]hexane hydrochloride.
1.4 Any of Method 1-15 or 1.1-1.3, wherein (1R,5S)-1-(3,4-dichlorophenyl)-3-azabicyclo[3.1.0]hexane in pharmaceutically acceptable salt form is crystalline.
1.5 Any of Method 1-15 or 1.1-1.4, wherein (1R,5S)-1-(3,4-dichlorophenyl)-3-azabicyclo[3.1.0]hexane, in free or pharmaceutically acceptable salt form, is Polymorph A, for example, Polymorph A substantially free of other polymorphic forms.
1.6 Any of Method 1-15 or 1.1-1.4, wherein (1R,5S)-1-(3,4-dichlorophenyl)-3-azabicyclo[3.1.0]hexane, in free or pharmaceutically acceptable salt form, is Polymorph B, for example, Polymorph B substantially free of other polymorphic forms.
1.7 Any of Method 1-15 or 1.1-1.4, wherein (1R,5S)-1-(3,4-dichlorophenyl)-3-azabicyclo[3.1.0]hexane, in free or pharmaceutically acceptable salt form, is Polymorph C, for example, Polymorph C substantially free of other polymorphic forms.
1.8 Any of Method 1-15 or 1.1-1.7, wherein the method comprises administering (1R,5S)-1-(3,4-dichlorophenyl)-3-azabicyclo[3.1.0]hexane, in free or pharmaceutically acceptable salt form, once, twice, three, or four times daily, e.g., once daily, e.g., twice daily, e.g., three times daily, e.g., four times daily.
1.9 Any of Method 1-6, 8-15, or 1.1-1.8, wherein (1R,5S)-1-(3,4-dichlorophenyl)-3-azabicyclo[3.1.0]hexane, in free or pharmaceutically acceptable salt form, is administered at least 1 week before target quit smoking date, e.g., 1 week before, e.g., 2 weeks before, e.g., 3 weeks before, e.g., 4 weeks before.
1.10 Any of Method 1-3, 5, 6, 8-15, or 1.1-1.9, wherein the method further comprises administering an effective amount of an NRT. For example, wherein the method further comprises administering an effective amount of an NRT at least 1 week before target quit smoking date, e.g., 1 week before, e.g., 2 weeks before, e.g., 3 weeks before, e.g., 4 weeks before.
1.11 Any of Method 1 or 1.1-1.10, wherein the human has previously been refractory to at least one (e.g., one or two or more) adequate prior course of approved treatment for nicotine dependency and nicotine withdrawal effects (e.g., at least one adequate prior course of approved treatment for smoking cessation) or wherein the human has relapsed after at least one adequate prior course of successful approved treatment for nicotine dependency and nicotine withdrawal effects (e.g., wherein the human has relapsed after at least one adequate prior course of successful approved treatment for smoking cessation) because the human is an intermediate, normal, or fast nicotine metabolizer (e.g., intermediate nicotine metabolizer or a normal nicotine metabolizer or a fast nicotine metabolizer).
1.12 Any of Method 1 or 1.1-1.11, wherein the human has previously been refractory to at least one adequate prior course of approved treatment for nicotine dependency and nicotine withdrawal effects (e.g., at least one adequate prior course of approved treatment for smoking cessation) or wherein the human has relapsed after at least one adequate prior course of successful approved treatment for nicotine dependency and nicotine withdrawal effects (e.g., wherein the human has relapsed after at least one adequate prior course of successful approved treatment for smoking cessation) because the human has a wildtype CYP2A6 allele or a CYP2A6 allele that produces a phenotype that increases rate of metabolism of nicotine compared to wildtype CYP2A6 (for instance, the human has an NMR indicative of an intermediate, a normal, or a fast nicotine metabolizer).
1.13 Any of Method 1 or 1.1-1.12, wherein the human has previously been refractory to at least one adequate prior course of approved treatment for nicotine dependency and nicotine withdrawal effects (e.g., at least one adequate prior course of approved treatment for smoking cessation) or wherein the human has relapsed after at least one adequate prior course of successful approved treatment for nicotine dependency and nicotine withdrawal effects (e.g., wherein the human has relapsed after at least one adequate prior course of successful approved treatment for smoking cessation) because the human is heterozygous with one decreased activity CYP2A6 allele and one active CYP2A6 allele (for instance, the human has an NMR indicative of an intermediate nicotine metabolizer). 1.14 Any of Method 1 or 1.1-1.12, wherein the human has previously been refractory to at least one adequate prior course of approved treatment for nicotine dependency and nicotine withdrawal effects (e.g., at least one adequate prior course of approved treatment for smoking cessation) or wherein the human has relapsed after at least one adequate prior course of successful approved treatment for nicotine dependency and nicotine withdrawal effects (e.g., wherein the human has relapsed after at least one adequate prior course of successful approved treatment for smoking cessation) because the human has 2 active CYP2A6 alleles (for instance, the human has an NMR indicative of a normal nicotine metabolizer).
1.15 Any of Method 1 or 1.1-1.12, wherein the human has previously been refractory to at least one adequate prior course of approved treatment for nicotine dependency and nicotine withdrawal effects (e.g., at least one adequate prior course of approved treatment for smoking cessation) or wherein the human has relapsed after at least one adequate prior course of successful approved treatment for nicotine dependency and nicotine withdrawal effects (e.g., wherein the human has relapsed after at least one adequate prior course of successful approved treatment for smoking cessation) because the human has >2 active CYP2A6 alleles (for instance, the human has an NMR indicative of a fast nicotine metabolizer).
1.16 Any of Method 1 or 1.1-1.15, wherein the at least one adequate prior course of approved treatment or the at least one adequate prior course of successful approved treatment for nicotine dependency and nicotine withdrawal effects is an adequate prior course of an NRT (e.g., a nicotine patch, gum, lozenge, inhaler, or nasal spray or oral nicotine (e.g., tablet or capsule)). For instance, an adequate prior course of a nicotine patch (e.g., Nicoderm® CQ®) (e.g., one 21 mg nicotine patch per day for weeks 1-6, one 14 mg nicotine patch per day for weeks 7-8, and one 7 mg nicotine patch per day for weeks 9-10 or one 14 mg nicotine patch per day for weeks 1-6 and one 7 mg nicotine patch per day for weeks 7-8). Or, for instance, an adequate prior course of nicotine gum or lozenge (e.g., for nicotine gum or lozenge one 2 mg or 4 mg piece or lozenge every 1 to 2 hours for weeks 1 to 6, one 2 mg or 4 mg piece or lozenge every 2 to 4 hours for weeks 7 to 9, and one 2 mg or 4 mg piece or lozenge every 4 to 8 hours for weeks 10-12).
1.17 Any of Method 1 or 1.1-1.16, wherein the at least one adequate prior course of approved treatment or the at least one adequate prior course of successful approved treatment for nicotine dependency and nicotine withdrawal effects is an adequate prior course of treatment of varenicline.
1.18 Any of Method 1 or 1.1-1.17, wherein the at least one adequate prior course of approved treatment or the at least one adequate prior course of successful approved treatment for nicotine dependency and nicotine withdrawal effects is an adequate prior course of treatment of bupropion.
1.19 Any of Method 1 or 1.1-1.18, wherein the at least one (e.g., one or two or more) adequate prior course of approved treatment or the at least one adequate prior course of successful approved treatment for nicotine dependency and nicotine withdrawal effects is a complete prior course of the treatment. For instance, bupropion or varenicline for 12 weeks or nicotine patch for 10 weeks or nicotine gum or lozenge for 12 weeks.
1.20 Any of Method 1 or 1.1-1.19, wherein the method comprises administering 75 mg to 200 mg of (1R,5S)-1-(3,4-dichlorophenyl)-3-azabicyclo[3.1.0]hexane, in free or pharmaceutically acceptable salt form, per day, for example, comprising administering 100 mg to 200 mg of (1R,5S)-1-(3,4-dichlorophenyl)-3-azabicyclo[3.1.0]hexane, in free or pharmaceutically acceptable salt form, per day, for example, comprising administering 150 mg of (1R,5S)-1-(3,4-dichlorophenyl)-3-azabicyclo[3.1.0]hexane, in free or pharmaceutically acceptable salt form, per day (e.g., by administering 75 mg twice per day, e.g., by administering three 25 mg tablets in the morning and three 25 mg tablets in the afternoon).
1.21 Any of Method or 1.1-1.20, wherein the method comprises administering 50 mg to 100 mg of (1R,5S)-1-(3,4-dichlorophenyl)-3-azabicyclo[3.1.0]hexane, in free or pharmaceutically acceptable salt form, twice daily, for example, comprising administering 75 mg of (1R,5S)-1-(3,4-dichlorophenyl)-3-azabicyclo[3.1.0]hexane, in free or pharmaceutically acceptable salt form, twice daily (e.g., by administering three 25 mg tablets in the morning and three 25 mg tablets in the afternoon).
1.22 Any of Method 1 or 1.1-1.21, wherein the method further comprises administering an effective amount of an NRT (e.g., an e-cigarette or “vape” or a nicotine patch, gum, lozenge, inhaler, or nasal spray or oral nicotine (e.g., tablet or capsule)) in combination with (1R,5S)-1-(3,4-dichlorophenyl)-3-azabicyclo[3.1.0]hexane, in free or pharmaceutically acceptable salt form.
1.23 Method 1.22, wherein the NRT is a nicotine patch (e.g., Nicoderm® CQ®.
1.24 Method 1.22 or 1.23, wherein the NRT is oral nicotine (e.g., oral nicotine delivered by tablet or capsule).
1.25 Any of Method 1.22-1.24, wherein the method comprises administering a subthreshold amount of the NRT (e.g., a nicotine patch, e.g., for a human that smokes more than 10 cigarettes per day, the nicotine patch starts with step 2 (i.e., one 14 mg nicotine patch per day) rather than step 1 (i.e., one 21 mg nicotine patch per day), so the human uses one 14 mg nicotine patch per day for weeks 1-6 and one 7 mg nicotine patch per day for weeks 7-8).
1.26 Any of Method 2, 9, 12, or 1.1-1.10 further comprising obtaining a biological sample (e.g., blood, plasma, saliva, and/or urine sample) from the human to determine whether the human is a poor, a slow, an intermediate, a normal, or a fast nicotine metabolizer (e.g., by measuring NMR).
1.27 Any of Method 2, 9, 12, 1.1-1.10, or 1.26, wherein the poor nicotine metabolizer has no active CYP2A6 alleles or one inactive and one decreased activity CYP2A6 alleles (for instance, the human has an NMR indicative of a poor nicotine metabolizer).
1.28 Any of Method 2, 9, 12, 1.1-1.10, or 1.26, wherein the slow nicotine metabolizer has one inactive or two decreased activity CYP2A6 alleles (for instance, the human has an NMR indicative of a slow nicotine metabolizer).
1.29 Any of Method 2, 9, 12, 1.1-1.10, or 1.26, wherein the intermediate nicotine metabolizer is heterozygous with one decreased activity CYP2A6 allele and one active CYP2A6 allele (for instance, the human has an NMR indicative of an intermediate nicotine metabolizer).
1.30 Any of Method 2, 9, 12, 1.1-1.10, or 1.26, wherein the normal nicotine metabolizer has two active CYP2A6 alleles (for instance, the human has an NMR indicative of a normal nicotine metabolizer).
1.31 Any of Method 2, 9, 12, 1.1-1.10, or 1.26, wherein the fast nicotine metabolizer has a CYP2A6 allele that produces a phenotype that increases rate of metabolism of nicotine compared to wildtype CYP2A6 (for instance, the human has an NMR indicative of a fast nicotine metabolizer).
1.32 Any of Method 2, 9, 12, 1.1-1.10, 1.26, or 1.31, wherein the fast nicotine metabolizer has greater than two active CYP2A6 alleles (for instance, the human has an NMR indicative of a fast nicotine metabolizer).
1.33 Any of Method 2, 9, 12, 1.1-1.10, or 1.26-1.32, wherein the method further comprises administering an effective amount of an NRT (e.g., an e-cigarette or “vape” or a nicotine patch, gum, lozenge, inhaler, or nasal spray or oral nicotine (e.g., tablet or capsule)) in combination with (1R,5S)-1-(3,4-dichlorophenyl)-3-azabicyclo[3.1.0]hexane, in free or pharmaceutically acceptable salt form. For instance, wherein the method further comprises administering an effective amount of an NRT at least 1 week before target quit smoking date, e.g., 1 week before, e.g., 2 weeks before, e.g., 3 weeks before, e.g., 4 weeks before.
1.34 Method 1.33, wherein the NRT is a nicotine patch (e.g., Nicoderm® CQ®) or oral nicotine (e.g., oral nicotine delivered by tablet or capsule) (e.g., the NRT is a nicotine patch) (e.g., the NRT is oral nicotine (e.g., oral nicotine delivered by tablet or capsule)).
1.35 Method 1.33 or 1.34, wherein the method comprises administering a subthreshold amount of the NRT (e.g., a nicotine patch, e.g., for a human that smokes more than 10 cigarettes per day, the nicotine patch starts with step 2 (i.e., one 14 mg nicotine patch per day) rather than step 1 (i.e., one 21 mg nicotine patch per day), so the human uses one 14 mg nicotine patch per day for weeks 1-6 and one 7 mg nicotine patch per day for weeks 7-8, or, e.g., the human uses one 7 mg nicotine patch per day for 8 weeks).
1.36 Any of Method 2, 9, 12, 1.1-1.10, 1.26, or 1.29-1.35, wherein the method comprises administering 75 mg to 200 mg of (1R,5S)-1-(3,4-dichlorophenyl)-3-azabicyclo[3.1.0]hexane, in free or pharmaceutically acceptable salt form, per day, for example, comprising administering 100 mg to 200 mg of (1R,5S)-1-(3,4-dichlorophenyl)-3-azabicyclo[3.1.0]hexane, in free or pharmaceutically acceptable salt form, per day, for example, comprising administering 150 mg of (1R,5S)-1-(3,4-dichlorophenyl)-3-azabicyclo[3.1.0]hexane, in free or pharmaceutically acceptable salt form, per day (e.g., by administering 75 mg twice per day, e.g., by administering three 25 mg tablets in the morning and three 25 mg tablets in the afternoon).
1.37 Method 1.36, wherein the method comprises administering 50 mg to 100 mg of (1R,5S)-1-(3,4-dichlorophenyl)-3-azabicyclo[3.1.0]hexane, in free or pharmaceutically acceptable salt form, twice daily, for example, comprising administering 75 mg of (1R,5S)-1-(3,4-dichlorophenyl)-3-azabicyclo[3.1.0]hexane, in free or pharmaceutically acceptable salt form, twice daily (e.g., by administering three 25 mg tablets in the morning and three 25 mg tablets in the afternoon).
1.38 Any of Method 2, 9, 12, 1.1-1.10, 1.26-1.28, or 1.33-1.35, wherein the method comprises administering 25 mg to 75 mg of (1R,5S)-1-(3,4-dichlorophenyl)-3-azabicyclo[3.1.0]hexane, in free or pharmaceutically acceptable salt form, per day, for example, comprising administering 25 mg, 50 mg, or 75 mg of (1R,5S)-1-(3,4-dichlorophenyl)-3-azabicyclo[3.1.0]hexane, in free or pharmaceutically acceptable salt form, per day (e.g., by administering 25 mg once or twice per day, e.g., by administering one 25 mg tablet in the morning and one 25 mg tablet in the afternoon).
1.39 Any of Method 2, 9, 12, 1.1-1.10, or 1.26-1.38, wherein the human has previously been refractory to at least one (e.g., one or two or more) adequate prior course of approved treatment for nicotine dependency and nicotine withdrawal effects (e.g., wherein the human has previously been refractory to at least one adequate prior course of approved treatment for smoking cessation (e.g., an NRT)).
1.40 Any of Method 2, 9, 12, 1.1-1.10, or 1.26-1.38, wherein the human has relapsed after at least one (e.g., one or two or more) adequate prior course of successful approved treatment for nicotine dependency and nicotine withdrawal effects (e.g., wherein the human has relapsed after at least one adequate prior course of successful approved treatment for smoking cessation (e.g., an NRT)) (e.g., wherein the human has previously been refractory, e.g., wherein the human has relapsed).
1.41 Any of Method 3 or 1.1-1.10, wherein the intermediate nicotine metabolizer is heterozygous with one decreased activity CYP2A6 allele and one active CYP2A6 allele (for instance, the human has an NMR indicative of an intermediate nicotine metabolizer).
1.42 Any of Method 3 or 1.1-1.10, wherein the normal nicotine metabolizer has two active CYP2A6 alleles (for instance, the human has an NMR indicative of a normal nicotine metabolizer).
1.43 Any of Method 3 or 1.1-1.10, wherein the fast nicotine metabolizer has a CYP2A6 allele that produces a phenotype that increases metabolism of nicotine compared to wildtype CYP2A6 (for instance, the human has an NMR indicative of a fast nicotine metabolizer).
1.44 Any of Method 3, 1.1-1.10, or 1.43, wherein the fast nicotine metabolizer has greater than two active CYP2A6 alleles (for instance, the human has an NMR indicative of a fast nicotine metabolizer).
1.45 Any of Method 3, 1.1-1.10, or 1.41-1.44, wherein the NRT is a nicotine patch (e.g., Nicoderm® CQ®) or oral nicotine (e.g., oral nicotine delivered by tablet or capsule) (e.g., the NRT is a nicotine patch) (e.g., the NRT is oral nicotine (e.g., oral nicotine delivered by tablet or capsule)). Also, any of Method 3, 1.1-1.10, or 1.41-1.44, wherein the NRT is administered at least 1 week before target quit smoking date, e.g., 1 week before, e.g., 2 weeks before, e.g., 3 weeks before, e.g., 4 weeks before.
1.46 Any of Method 3, 1.1-1.10, or 1.41-1.45, wherein the method comprises administering 75 mg to 200 mg of (1R,5S)-1-(3,4-dichlorophenyl)-3-azabicyclo[3.1.0]hexane, in free or pharmaceutically acceptable salt form, per day, for example, comprising administering 100 mg to 200 mg of (1R,5S)-1-(3,4-dichlorophenyl)-3-azabicyclo[3.1.0]hexane, in free or pharmaceutically acceptable salt form, per day, for example, comprising administering 150 mg of (1R,5S)-1-(3,4-dichlorophenyl)-3-azabicyclo[3.1.0]hexane, in free or pharmaceutically acceptable salt form, per day (e.g., by administering 75 mg twice per day, e.g., by administering three 25 mg tablets in the morning and three 25 mg tablets in the afternoon).
1.47 Method 1.46, wherein the method comprises administering 50 mg to 100 mg of (1R,5S)-1-(3,4-dichlorophenyl)-3-azabicyclo[3.1.0]hexane, in free or pharmaceutically acceptable salt form, twice daily, for example, comprising administering 75 mg of (1R,5S)-1-(3,4-dichlorophenyl)-3-azabicyclo[3.1.0]hexane, in free or pharmaceutically acceptable salt form, twice daily (e.g., by administering three 25 mg tablets in the morning and three 25 mg tablets in the afternoon).
1.48 Any of Method 3, 1.1-1.10, or 1.41-1.47, wherein the method comprises administering a subthreshold amount of the NRT (e.g., a nicotine patch, e.g., for a human that smokes more than 10 cigarettes per day, the nicotine patch starts with step 2 (i.e., one 14 mg nicotine patch per day) rather than step 1 (i.e., one 21 mg nicotine patch per day), so the human uses one 14 mg nicotine patch per day for weeks 1-6 and one 7 mg nicotine patch per day for weeks 7-8).
1.49 Any of Method 4 or 1.1-1.10, wherein the poor nicotine metabolizer has no active CYP2A6 alleles or one inactive and one decreased activity CYP2A6 alleles (for instance, the human has an NMR indicative of a poor nicotine metabolizer).
1.50 Any of Method 4 or 1.1-1.10, wherein the slow nicotine metabolizer has one inactive or two decreased activity CYP2A6 alleles (for instance, the human has an NMR indicative of a slow nicotine metabolizer).
1.51 Any of Method 4, 1.1-1.10, 1.49, or 1.50, wherein the method comprises administering 25 mg to 75 mg of (1R,5S)-1-(3,4-dichlorophenyl)-3-azabicyclo[3.1.0]hexane, in free or pharmaceutically acceptable salt form, per day, for example, comprising administering 25 mg, 50 mg, or 75 mg of (1R,5S)-1-(3,4-dichlorophenyl)-3-azabicyclo[3.1.0]hexane, in free or pharmaceutically acceptable salt form, per day (e.g., by administering 25 mg once or twice per day, e.g., by administering one 25 mg tablet in the morning and one 25 mg tablet in the afternoon).
1.52 Any of Method 5, 5a, or 1.1-1.10, wherein the human is an intermediate, a normal, or a fast nicotine metabolizer.
1.53 Any of Method 5, 5a, 1.1-1.10, or 1.52, wherein the intermediate nicotine metabolizer is heterozygous with one decreased activity CYP2A6 allele and one active CYP2A6 allele (for instance, the human has an NMR indicative of an intermediate nicotine metabolizer).
1.54 Any of Method 5, 5a, 1.1-1.10, or 1.52, wherein the normal nicotine metabolizer has two active CYP2A6 alleles (for instance, the human has an NMR indicative of a normal nicotine metabolizer).
1.55 Any of Method 5, 5a, 1.1-1.10, or 1.52, wherein the fast nicotine metabolizer has a CYP2A6 allele that produces a phenotype that increases rate of metabolism of nicotine compared to wildtype CYP2A6 (for instance, the human has an NMR indicative of a fast nicotine metabolizer).
1.56 Any of Method 5, 5a, 1.1-1.10, 1.52, or 1.55, wherein the fast nicotine metabolizer has greater than two active CYP2A6 alleles (for instance, the human has an NMR indicative of a fast nicotine metabolizer).
1.57 Any of Method 5, 5a, 1.1-1.10, or 1.52-1.56, wherein the method comprises administering 75 mg to 200 mg of (1R,5S)-1-(3,4-dichlorophenyl)-3-azabicyclo[3.1.0]hexane, in free or pharmaceutically acceptable salt form, per day, for example, comprising administering 100 mg to 200 mg of (1R,5S)-1-(3,4-dichlorophenyl)-3-azabicyclo[3.1.0]hexane, in free or pharmaceutically acceptable salt form, per day, for example, comprising administering 150 mg of (1R,5S)-1-(3,4-dichlorophenyl)-3-azabicyclo[3.1.0]hexane, in free or pharmaceutically acceptable salt form, per day (e.g., by administering 75 mg twice per day, e.g., by administering three 25 mg tablets in the morning and three 25 mg tablets in the afternoon).
1.58 Method 1.57, wherein the method comprises administering 50 mg to 100 mg of (1R,5S)-1-(3,4-dichlorophenyl)-3-azabicyclo[3.1.0]hexane, in free or pharmaceutically acceptable salt form, twice daily, for example, comprising administering 75 mg of (1R,5S)-1-(3,4-dichlorophenyl)-3-azabicyclo[3.1.0]hexane, in free or pharmaceutically acceptable salt form, twice daily (e.g., by administering three 25 mg tablets in the morning and three 25 mg tablets in the afternoon).
1.59 Any of Method 5, 5a, 1.1-1.10, or 1.52-1.58, wherein the method further comprises administering an effective amount of an NRT (e.g., an e-cigarette or “vape” or a nicotine patch, gum, lozenge, inhaler, or nasal spray or oral nicotine (e.g., tablet or capsule)) in combination with (1R,5S)-1-(3,4-dichlorophenyl)-3-azabicyclo[3.1.0]hexane, in free or pharmaceutically acceptable salt form. For instance, wherein the method further comprises administering an effective amount of an NRT at least 1 week before target quit smoking date, e.g., 1 week before, e.g., 2 weeks before, e.g., 3 weeks before, e.g., 4 weeks before.
1.60 Method 1.59, wherein the NRT is a nicotine patch (e.g., Nicoderm® CQ®) or oral nicotine (e.g., oral nicotine delivered by tablet or capsule) (e.g., the NRT is a nicotine patch) (e.g., the NRT is oral nicotine (e.g., oral nicotine delivered by tablet or capsule)).
1.61 Method 1.59 or 1.60, wherein the method comprises administering a subthreshold amount of the NRT (e.g., a nicotine patch, e.g., for a human that smokes more than 10 cigarettes per day, the nicotine patch starts with step 2 (i.e., one 14 mg nicotine patch per day) rather than step 1 (i.e., one 21 mg nicotine patch per day), so the human uses one 14 mg nicotine patch per day for weeks 1-6 and one 7 mg nicotine patch per day for weeks 7-8).
1.62 Any of Method 6 or 1.1-1.10, wherein the intermediate nicotine metabolizer is heterozygous with one decreased activity CYP2A6 allele and one active CYP2A6 allele (for instance, the human has an NMR indicative of an intermediate nicotine metabolizer).
1.63 Any of Method 6 or 1.1-1.10, wherein the normal nicotine metabolizer has two active CYP2A6 alleles (for instance, the human has an NMR indicative of a normal nicotine metabolizer).
1.64 Any of Method 6 or 1.1-1.10, wherein the fast nicotine metabolizer has a CYP2A6 allele that produces a phenotype that increases rate of metabolism of nicotine compared to wildtype CYP2A6 (for instance, the human has an NMR indicative of a fast nicotine metabolizer).
1.65 Any of Method 6, 1.1-1.10, or 1.64, wherein the fast nicotine metabolizer has greater than two active CYP2A6 alleles (for instance, the human has an NMR indicative of a fast nicotine metabolizer).
1.66 Any of Method 6, 1.1-1.10, or 1.62-1.65, wherein the NRT is a nicotine patch (e.g., Nicoderm® CQ®) or oral nicotine (e.g., oral nicotine delivered by tablet or capsule) (e.g., the NRT is a nicotine patch) (e.g., the NRT is oral nicotine (e.g., oral nicotine delivered by tablet or capsule)). Also, any of Method 6, 1.1-1.10, or 1.62-1.65, wherein the NRT is administered at least 1 week before target quit smoking date, e.g., 1 week before, e.g., 2 weeks before, e.g., 3 weeks before, e.g., 4 weeks before.
1.67 Any of Method 6, 1.1-1.10, or 1.62-1.66, wherein the method comprises administering 75 mg to 200 mg of (1R,5S)-1-(3,4-dichlorophenyl)-3-azabicyclo[3.1.0]hexane, in free or pharmaceutically acceptable salt form, per day, for example, comprising administering 100 mg to 200 mg of (1R,5S)-1-(3,4-dichlorophenyl)-3-azabicyclo[3.1.0]hexane, in free or pharmaceutically acceptable salt form, per day, for example, comprising administering 150 mg of (1R,5S)-1-(3,4-dichlorophenyl)-3-azabicyclo[3.1.0]hexane, in free or pharmaceutically acceptable salt form, per day (e.g., by administering 75 mg twice per day, e.g., by administering three 25 mg tablets in the morning and three 25 mg tablets in the afternoon).
1.68 Method 1.67, wherein the method comprises administering 50 mg to 100 mg of (1R,5S)-1-(3,4-dichlorophenyl)-3-azabicyclo[3.1.0]hexane, in free or pharmaceutically acceptable salt form, twice daily, for example, comprising administering 75 mg of (1R,5S)-1-(3,4-dichlorophenyl)-3-azabicyclo[3.1.0]hexane, in free or pharmaceutically acceptable salt form, twice daily (e.g., by administering three 25 mg tablets in the morning and three 25 mg tablets in the afternoon).
1.69 Any of Method 6, 1.1-1.10, or 1.62-1.68, wherein the method comprises administering a subthreshold amount of the NRT (e.g., a nicotine patch, e.g., for a human that smokes more than 10 cigarettes per day, the nicotine patch starts with step 2 (i.e., one 14 mg nicotine patch per day) rather than step 1 (i.e., one 21 mg nicotine patch per day), so the human uses one 14 mg nicotine patch per day for weeks 1-6 and one 7 mg nicotine patch per day for weeks 7-8).
1.70 Any of Method 8 or 1.1-1.10, wherein the intermediate nicotine metabolizer is heterozygous with one decreased activity CYP2A6 allele and one active CYP2A6 allele (for instance, the human has an NMR indicative of an intermediate nicotine metabolizer).
1.71 Any of Method 8 or 1.1-1.10, wherein the normal nicotine metabolizer has two active CYP2A6 alleles (for instance, the human has an NMR indicative of a normal nicotine metabolizer).
1.72 Any of Method 8 or 1.1-1.10, wherein the fast nicotine metabolizer has a CYP2A6 allele that produces a phenotype that increases rate of metabolism of nicotine compared to wildtype CYP2A6 (for instance, the human has an NMR indicative of a fast nicotine metabolizer).
1.73 Any of Method 8, 1.1-1.10, or 1.72, wherein the fast nicotine metabolizer has greater than two active CYP2A6 alleles (for instance, the human has an NMR indicative of a fast nicotine metabolizer).
1.74 Any of Method 8, 1.1-1.10, or 1.70-1.73, wherein the method comprises administering 75 mg to 200 mg of (1R,5S)-1-(3,4-dichlorophenyl)-3-azabicyclo[3.1.0]hexane, in free or pharmaceutically acceptable salt form, per day, for example comprising administering 100 mg to 200 mg of (1R,5S)-1-(3,4-dichlorophenyl)-3-azabicyclo[3.1.0]hexane, in free or pharmaceutically acceptable salt form, per day, for example, comprising administering 150 mg of (1R,5S)-1-(3,4-dichlorophenyl)-3-azabicyclo[3.1.0]hexane, in free or pharmaceutically acceptable salt form, per day (e.g., by administering 75 mg twice per day, e.g., by administering three 25 mg tablets in the morning and three 25 mg tablets in the afternoon).
1.75 Method 1.74, wherein the method comprises administering 50 mg to 100 mg of (1R,5S)-1-(3,4-dichlorophenyl)-3-azabicyclo[3.1.0]hexane, in free or pharmaceutically acceptable salt form, twice daily, for example, comprising administering 75 mg of (1R,5S)-1-(3,4-dichlorophenyl)-3-azabicyclo[3.1.0]hexane, in free or pharmaceutically acceptable salt form, twice daily (e.g., by administering three 25 mg tablets in the morning and three 25 mg tablets in the afternoon).
1.76 Any of Method 8, 1.1-1.10, or 1.70-1.75, wherein the method further comprises administering an effective amount of an NRT (e.g., an e-cigarette or “vape” or a nicotine patch, gum, lozenge, inhaler, or nasal spray or oral nicotine (e.g., tablet or capsule)) in combination with (1R,5S)-1-(3,4-dichlorophenyl)-3-azabicyclo[3.1.0]hexane, in free or pharmaceutically acceptable salt form. For instance, wherein the method further comprises administering an effective amount of an NRT at least 1 week before target quit smoking date, e.g., 1 week before, e.g., 2 weeks before, e.g., 3 weeks before, e.g., 4 weeks before.
1.77 Method 1.76, wherein the NRT is a nicotine patch (e.g., Nicoderm® CQ®.
1.78 Method 1.76 or 1.77, wherein the method comprises administering a subthreshold amount of the NRT (e.g., a nicotine patch, e.g., for a human that smokes more than 10 cigarettes per day, the nicotine patch starts with step 2 (i.e., one 14 mg nicotine patch per day) rather than step 1 (i.e., one 21 mg nicotine patch per day), so the human uses one 14 mg nicotine patch per day for weeks 1-6 and one 7 mg nicotine patch per day for weeks 7-8).
1.79 Any of Method 8, 1.1-1.10, or 1.70-1.78, wherein the one or more withdrawal effects are one or more of quit-related changes in mood (e.g., depression and/or anxiety), anhedonia, cue-associated impulsive loss of control, and weight change (e.g., quit-related depression and/or quit-related anxiety and/or anhedonia and/or cue-associated impulsive loss of control and/or weight change and/or weight gain).
1.80 Any of Method 1-4, 9, 10, 15, 1.1-1.51, 1.81-1.90, or 1.116-1.146, wherein the one or more withdrawal effects are one or more of quit-related changes in mood (e.g., depression and/or anxiety), anhedonia, cue-associated impulsive loss of control, and weight change (e.g., quit-related depression and/or quit-related anxiety and/or anhedonia and/or cue-associated impulsive loss of control and/or weight change and/or weight gain).
1.81 Any of Method 10 or 1.1-1.10, wherein the human is an intermediate, a normal, or a fast nicotine metabolizer.
1.82 Method 1.81, wherein the intermediate nicotine metabolizer is heterozygous with one decreased activity CYP2A6 allele and one active CYP2A6 allele (for instance, the human has an NMR indicative of an intermediate nicotine metabolizer).
1.83 Method 1.81, wherein the normal nicotine metabolizer has two active CYP2A6 alleles (for instance, the human has an NMR indicative of a normal nicotine metabolizer).
1.84 Method 1.81, wherein the fast nicotine metabolizer has a CYP2A6 allele that produces a phenotype that increases rate of metabolism of nicotine compared to wildtype CYP2A6 (for instance, the human has an NMR indicative of a fast nicotine metabolizer).
1.85 Method 1.81 or 1.84, wherein the fast nicotine metabolizer has greater than two active CYP2A6 alleles (for instance, the human has an NMR indicative of a fast nicotine metabolizer).
1.86 Any of Method 10, 1.1-1.10, or 1.81-1.85, wherein the method comprises administering 75 mg to 200 mg of (1R,5S)-1-(3,4-dichlorophenyl)-3-azabicyclo[3.1.0]hexane, in free or pharmaceutically acceptable salt form, per day, for example, comprising administering 100 mg to 200 mg of (1R,5S)-1-(3,4-dichlorophenyl)-3-azabicyclo[3.1.0]hexane, in free or pharmaceutically acceptable salt form, per day, for example, comprising administering 150 mg of (1R,5S)-1-(3,4-dichlorophenyl)-3-azabicyclo[3.1.0]hexane, in free or pharmaceutically acceptable salt form, per day (e.g., by administering 75 mg twice per day, e.g., by administering three 25 mg tablets in the morning and three 25 mg tablets in the afternoon).
1.87 Method 1.86, wherein the method comprises administering 50 mg to 100 mg of (1R,5S)-1-(3,4-dichlorophenyl)-3-azabicyclo[3.1.0]hexane, in free or pharmaceutically acceptable salt form, twice daily, for example, comprising administering 75 mg of (1R,5S)-1-(3,4-dichlorophenyl)-3-azabicyclo[3.1.0]hexane, in free or pharmaceutically acceptable salt form, twice daily (e.g., by administering three 25 mg tablets in the morning and three 25 mg tablets in the afternoon).
1.88 Any of Method 10, 1.1-1.10, or 1.81-1.87, wherein the method further comprises administering an effective amount of an NRT (e.g., an e-cigarette or “vape” or a nicotine patch, gum, lozenge, inhaler, or nasal spray or oral nicotine (e.g., tablet or capsule)) in combination with (1R,5S)-1-(3,4-dichlorophenyl)-3-azabicyclo[3.1.0]hexane, in free or pharmaceutically acceptable salt form. For instance, wherein the method further comprises administering an effective amount of an NRT at least 1 week before target quit smoking date, e.g., 1 week before, e.g., 2 weeks before, e.g., 3 weeks before, e.g., 4 weeks before.
1.89 Method 1.88, wherein the NRT is a nicotine patch (e.g., Nicoderm® CQ®) or oral nicotine (e.g., oral nicotine delivered by tablet or capsule) (e.g., the NRT is a nicotine patch) (e.g., the NRT is oral nicotine (e.g., oral nicotine delivered by tablet or capsule)).
1.90 Method 1.88 or 1.89, wherein the method comprises administering a subthreshold amount of the NRT (e.g., a nicotine patch, e.g., for a human that smokes more than 10 cigarettes per day, the nicotine patch starts with step 2 (i.e., one 14 mg nicotine patch per day) rather than step 1 (i.e., one 21 mg nicotine patch per day), so the human uses one 14 mg nicotine patch per day for weeks 1-6 and one 7 mg nicotine patch per day for weeks 7-8).
1.91 Any of Method 11 or 1.1-1.10, wherein the intermediate nicotine metabolizer is heterozygous with one decreased activity CYP2A6 allele and one active CYP2A6 allele (for instance, the human has an NMR indicative of an intermediate nicotine metabolizer).
1.92 Any of Method 11 or 1.1-1.10, wherein the normal nicotine metabolizer has two active CYP2A6 alleles (for instance, the human has an NMR indicative of a normal nicotine metabolizer).
1.93 Any of Method 11 or 1.1-1.10, wherein the fast nicotine metabolizer has a CYP2A6 allele that produces a phenotype that increases rate of metabolism of nicotine compared to wildtype CYP2A6 (for instance, the human has an NMR indicative of a fast nicotine metabolizer).
1.94 Any of Method 11, 1.1-1.10, or 1.93, wherein the fast nicotine metabolizer has greater than two active CYP2A6 alleles (for instance, the human has an NMR indicative of a fast nicotine metabolizer).
1.95 Any of Method 11, 1.1-1.10, or 1.91-1.94, wherein the method comprises administering 75 mg to 200 mg of (1R,5S)-1-(3,4-dichlorophenyl)-3-azabicyclo[3.1.0]hexane, in free or pharmaceutically acceptable salt form, per day, for example, comprising administering 100 mg to 200 mg of (1R,5S)-1-(3,4-dichlorophenyl)-3-azabicyclo[3.1.0]hexane, in free or pharmaceutically acceptable salt form, per day, for example, comprising administering 150 mg of (1R,5S)-1-(3,4-dichlorophenyl)-3-azabicyclo[3.1.0]hexane, in free or pharmaceutically acceptable salt form, per day (e.g., by administering 75 mg twice per day, e.g., by administering three 25 mg tablets in the morning and three 25 mg tablets in the afternoon).
1.96 Method 1.95, wherein the method comprises administering 50 mg to 100 mg of (1R,5S)-1-(3,4-dichlorophenyl)-3-azabicyclo[3.1.0]hexane, in free or pharmaceutically acceptable salt form, twice daily, for example, comprising administering 75 mg of (1R,5S)-1-(3,4-dichlorophenyl)-3-azabicyclo[3.1.0]hexane, in free or pharmaceutically acceptable salt form, twice daily (e.g., by administering three 25 mg tablets in the morning and three 25 mg tablets in the afternoon).
1.97 Any of Method 11, 1.1-1.10, or 1.91-1.96, wherein the method further comprises administering an effective amount of an NRT (e.g., an e-cigarette or “vape” or a nicotine patch, gum, lozenge, inhaler, or nasal spray or oral nicotine (e.g., tablet or capsule)) in combination with (1R,5S)-1-(3,4-dichlorophenyl)-3-azabicyclo[3.1.0]hexane, in free or pharmaceutically acceptable salt form. For instance, wherein the method further comprises administering an effective amount of an NRT at least 1 week before target quit smoking date, e.g., 1 week before, e.g., 2 weeks before, e.g., 3 weeks before, e.g., 4 weeks before.
1.98 Method 1.97, wherein the NRT is a nicotine patch (e.g., Nicoderm® CQ®) or oral nicotine (e.g., oral nicotine delivered by tablet or capsule) (e.g., the NRT is a nicotine patch) (e.g., the NRT is oral nicotine (e.g., oral nicotine delivered by tablet or capsule)).
1.99 Method 1.97 or 1.98, wherein the method comprises administering a subthreshold amount of the NRT (e.g., a nicotine patch, e.g., for a human that smokes more than 10 cigarettes per day, the nicotine patch starts with step 2 (i.e., one 14 mg nicotine patch per day) rather than step 1 (i.e., one 21 mg nicotine patch per day), so the human uses one 14 mg nicotine patch per day for weeks 1-6 and one 7 mg nicotine patch per day for weeks 7-8).
1.100 Any of Method 13 or 1.1-1.10, wherein the human is a poor or slow nicotine metabolizer.
1.101 Any of Method 13 or 1.1-1.10, wherein the human is an intermediate, a normal, or a fast nicotine metabolizer.
1.102 Method 1.100 or 1.101 further comprising obtaining a biological sample (e.g., blood, plasma, saliva, and/or urine sample) from the human to determine whether the human is a poor, a slow, an intermediate, a normal, or a fast nicotine metabolizer (e.g., by measuring NMR).
1.103 Method 1.100 or 1.102, wherein the poor nicotine metabolizer has no active CYP2A6 alleles or one inactive and one decreased activity CYP2A6 alleles (for instance, the human has an NMR indicative of a poor nicotine metabolizer).
1.104 Method 1.100 or 1.102, wherein the slow nicotine metabolizer has one inactive or two decreased activity CYP2A6 alleles (for instance, the human has an NMR indicative of a slow nicotine metabolizer).
1.105 Method 1.101 or 1.102, wherein the intermediate nicotine metabolizer is heterozygous with one decreased activity CYP2A6 allele and one active CYP2A6 allele (for instance, the human has an NMR indicative of an intermediate nicotine metabolizer).
1.106 Method 1.101 or 1.102, wherein the normal nicotine metabolizer has two active CYP2A6 alleles (for instance, the human has an NMR indicative of a normal nicotine metabolizer).
1.107 Method 1.101 or 1.102, wherein the fast nicotine metabolizer has a CYP2A6 allele that produces a phenotype that increases rate of metabolism of nicotine compared to wildtype CYP2A6 (for instance, the human has an NMR indicative of a fast nicotine metabolizer).
1.108 Any of Method 1.101, 1.102, or 1.107, wherein the fast nicotine metabolizer has greater than two active CYP2A6 alleles (for instance, the human has an NMR indicative of a fast nicotine metabolizer).
1.109 Any of Method 13, 1.1-1.10, or 1.100-1.108, wherein the method further comprises administering an effective amount of an NRT (e.g., an e-cigarette or “vape” or a nicotine patch, gum, lozenge, inhaler, or nasal spray or oral nicotine (e.g., tablet or capsule)) in combination with (1R,5S)-1-(3,4-dichlorophenyl)-3-azabicyclo[3.1.0]hexane, in free or pharmaceutically acceptable salt form. For instance, wherein the method further comprises administering an effective amount of an NRT at least 1 week before target quit smoking date, e.g., 1 week before, e.g., 2 weeks before, e.g., 3 weeks before, e.g., 4 weeks before.
1.110 Method 1.109, wherein the NRT is a nicotine patch (e.g., Nicoderm® CQ®.
1.111 Method 1.109, wherein the NRT is oral nicotine (e.g., oral nicotine delivered by tablet or capsule).
1.112 Any of Method 1.109-1.111, wherein the method comprises administering a subthreshold amount of the NRT (e.g., a nicotine patch, e.g., for a human that smokes more than 10 cigarettes per day, the nicotine patch starts with step 2 (i.e., one 14 mg nicotine patch per day) rather than step 1 (i.e., one 21 mg nicotine patch per day), so the human uses one 14 mg nicotine patch per day for weeks 1-6 and one 7 mg nicotine patch per day for weeks 7-8 or, e.g., the human uses one 7 mg nicotine patch per day for 8 weeks).
1.113 Any of Method 13, 1.1-1.10, 1.101, 1.102, or 1.105-1.112, wherein the method comprises administering 75 mg to 200 mg of (1R,5S)-1-(3,4-dichlorophenyl)-3-azabicyclo[3.1.0]hexane, in free or pharmaceutically acceptable salt form, per day, for example, comprising administering 100 mg to 200 mg of (1R,5S)-1-(3,4-dichlorophenyl)-3-azabicyclo[3.1.0]hexane, in free or pharmaceutically acceptable salt form, per day, for example, comprising administering 150 mg of (1R,5S)-1-(3,4-dichlorophenyl)-3-azabicyclo[3.1.0]hexane, in free or pharmaceutically acceptable salt form, per day (e.g., by administering 75 mg twice per day, e.g., by administering three 25 mg tablets in the morning and three 25 mg tablets in the afternoon).
1.114 Method 1.113, wherein the method comprises administering 50 mg to 100 mg of (1R,5S)-1-(3,4-dichlorophenyl)-3-azabicyclo[3.1.0]hexane, in free or pharmaceutically acceptable salt form, twice daily, for example, comprising administering 75 mg of (1R,5S)-1-(3,4-dichlorophenyl)-3-azabicyclo[3.1.0]hexane, in free or pharmaceutically acceptable salt form, twice daily (e.g., by administering three 25 mg tablets in the morning and three 25 mg tablets in the afternoon).
1.115 Any of Method 13, 1.1-1.10, 1.100, 1.102-1.104, or 1.109-1.112, wherein the method comprises administering 25 mg to 75 mg of (1R,5S)-1-(3,4-dichlorophenyl)-3-azabicyclo[3.1.0]hexane, in free or pharmaceutically acceptable salt form, per day, for example, comprising administering 25 mg, 50 mg, or 75 mg of (1R,5S)-1-(3,4-dichlorophenyl)-3-azabicyclo[3.1.0]hexane, in free or pharmaceutically acceptable salt form, per day (e.g., by administering 25 mg once or twice per day, e.g., by administering one 25 mg tablet in the morning and one 25 mg tablet in the afternoon).
1.116 Any of Method 14, 15, or 1.1-1.10, wherein the human (e.g., human smoker) is a poor or slow nicotine metabolizer.
1.117 Any of Method 14, 15, or 1.1-1.10, wherein the human (e.g., human smoker) is an intermediate, a normal, or a fast nicotine metabolizer.
1.118 Method 1.116 or 1.117 further comprising obtaining a biological sample (e.g., blood, plasma, saliva, and/or urine sample) from the human to determine whether the human is a poor, a slow, an intermediate, a normal, or a fast nicotine metabolizer (e.g., by measuring NMR).
1.119 Method 1.116 or 1.118, wherein the poor nicotine metabolizer has no active CYP2A6 alleles or one inactive and one decreased activity CYP2A6 alleles (for instance, the human has an NMR indicative of a poor nicotine metabolizer).
1.120 Method 1.116 or 1.118, wherein the slow nicotine metabolizer has one inactive or two decreased activity CYP2A6 alleles (for instance, the human has an NMR indicative of a slow nicotine metabolizer).
1.121 Method 1.117 or 1.118, wherein the intermediate nicotine metabolizer is heterozygous with one decreased activity CYP2A6 allele and one active CYP2A6 allele (for instance, the human has an NMR indicative of an intermediate nicotine metabolizer).
1.122 Method 1.117 or 1.118, wherein the normal nicotine metabolizer has two active CYP2A6 alleles (for instance, the human has an NMR indicative of a normal nicotine metabolizer).
1.123 Method 1.117 or 1.118, wherein the fast nicotine metabolizer has a CYP2A6 allele that produces a phenotype that increases rate of metabolism of nicotine compared to wildtype CYP2A6 (for instance, the human has an NMR indicative of a fast nicotine metabolizer).
1.124 Any of Method 1.117, 1.118, or 1.123, wherein the fast nicotine metabolizer has greater than two active CYP2A6 alleles (for instance, the human has an NMR indicative of a fast nicotine metabolizer).
1.125 Any of Method 14, 15, 1.1-1.10, or 1.116-1.124, wherein the method further comprises administering an effective amount of a second NRT (e.g., an e-cigarette or “vape” or a nicotine patch, gum, lozenge, inhaler, or nasal spray or oral nicotine (e.g., tablet or capsule)) in combination with (1R,5S)-1-(3,4-dichlorophenyl)-3-azabicyclo[3.1.0]hexane, in free or pharmaceutically acceptable salt form.
1.126 Method 1.125, wherein the second NRT is a nicotine patch (e.g., Nicoderm® CQ®.
1.127 Method 1.125 or 1.126, wherein the method comprises administering a subthreshold amount of the second NRT (e.g., a nicotine patch, e.g., for a human that smokes more than 10 cigarettes per day, the nicotine patch starts with step 2 (i.e., one 14 mg nicotine patch per day) rather than step 1 (i.e., one 21 mg nicotine patch per day), so the human uses one 14 mg nicotine patch per day for weeks 1-6 and one 7 mg nicotine patch per day for weeks 7-8 or, e.g., the human uses one 7 mg nicotine patch per day for 8 weeks).
1.128 Any of Method 1.125-1.127, wherein the method comprises administering the second NRT (e.g., an e-cigarette or “vape” or a nicotine patch, gum, lozenge, inhaler, or nasal spray or oral nicotine (e.g., tablet or capsule), e.g., a nicotine patch) at least 1 week before target quit smoking date, e.g., 1 week before, e.g., 2 weeks before, e.g., 3 weeks before, e.g., 4 weeks before.
1.129 Any of Method 14, 15, 1.1-1.10, 1.117, 1.118, or 1.121-1.128, wherein the method comprises administering 75 mg to 200 mg of (1R,5S)-1-(3,4-dichlorophenyl)-3-azabicyclo[3.1.0]hexane, in free or pharmaceutically acceptable salt form, per day, for example, comprising administering 100 mg to 200 mg of (1R,5S)-1-(3,4-dichlorophenyl)-3-azabicyclo[3.1.0]hexane, in free or pharmaceutically acceptable salt form, per day, for example, comprising administering 150 mg of (1R,5S)-1-(3,4-dichlorophenyl)-3-azabicyclo[3.1.0]hexane, in free or pharmaceutically acceptable salt form, per day (e.g., by administering 75 mg twice per day, e.g., by administering three 25 mg tablets in the morning and three 25 mg tablets in the afternoon).
1.130 Method 1.129, wherein the method comprises administering 50 mg to 100 mg of (1R,5S)-1-(3,4-dichlorophenyl)-3-azabicyclo[3.1.0]hexane, in free or pharmaceutically acceptable salt form, twice daily, for example, comprising administering 75 mg of (1R,5S)-1-(3,4-dichlorophenyl)-3-azabicyclo[3.1.0]hexane, in free or pharmaceutically acceptable salt form, twice daily (e.g., by administering three 25 mg tablets in the morning and three 25 mg tablets in the afternoon).
1.131 Any of Method 14, 15, 1.1-1.10, 1.116, 1.118-1.120, or 1.125-1.128, wherein the method comprises administering 25 mg to 75 mg of (1R,5S)-1-(3,4-dichlorophenyl)-3-azabicyclo[3.1.0]hexane, in free or pharmaceutically acceptable salt form, per day, for example, comprising administering 25 mg, 50 mg, or 75 mg of (1R,5S)-1-(3,4-dichlorophenyl)-3-azabicyclo[3.1.0]hexane, in free or pharmaceutically acceptable salt form, per day (e.g., by administering 25 mg once or twice per day, e.g., by administering one 25 mg tablet in the morning and one 25 mg tablet in the afternoon).
1.132 Any of Method 14, 15, 1.1-1.10, or 1.116-1.131, wherein the method comprises administering a subthreshold amount of the oral nicotine (e.g., tablet or capsule).
1.133 Any of Method 14, 15, 1.1-1.10, or 1.116-1.132, wherein the method comprises administering the oral nicotine (e.g., tablet or capsule) at least 1 week before target quit smoking date, e.g., 1 week before, e.g., 2 weeks before, e.g., 3 weeks before, e.g., 4 weeks before.
1.134 Any of Method 5, 5a, 10, or 1.1-1.10, wherein the human is a poor or slow nicotine metabolizer.
1.135 Method 1.134, wherein the poor nicotine metabolizer has no active CYP2A6 alleles or one inactive and one decreased activity CYP2A6 alleles (for instance, the human has an NMR indicative of a poor nicotine metabolizer).
1.136 Method 1.134, wherein the slow nicotine metabolizer has one inactive or two decreased activity CYP2A6 alleles (for instance, the human has an NMR indicative of a slow nicotine metabolizer).
1.137 Any of Method 5, 5a, 10, 1.1-1.10, or 1.134-1.136, wherein the method comprises administering 25 mg to 75 mg of (1R,5S)-1-(3,4-dichlorophenyl)-3-azabicyclo[3.1.0]hexane, in free or pharmaceutically acceptable salt form, per day, for example, comprising administering 25 mg, 50 mg, or 75 mg of (1R,5S)-1-(3,4-dichlorophenyl)-3-azabicyclo[3.1.0]hexane, in free or pharmaceutically acceptable salt form, per day (e.g., by administering 25 mg once or twice per day, e.g., by administering one 25 mg tablet in the morning and one 25 mg tablet in the afternoon).
1.138 Any of Method 5, 5a, 10, 1.1-1.10, or 1.134-1.137, wherein the method further comprises administering an effective amount of an NRT (e.g., an e-cigarette or “vape” or a nicotine patch, gum, lozenge, inhaler, or nasal spray or oral nicotine (e.g., tablet or capsule)) in combination with (1R,5S)-1-(3,4-dichlorophenyl)-3-azabicyclo[3.1.0]hexane, in free or pharmaceutically acceptable salt form. For instance, wherein the method further comprises administering an effective amount of an NRT at least 1 week before target quit smoking date, e.g., 1 week before, e.g., 2 weeks before, e.g., 3 weeks before, e.g., 4 weeks before.
1.139 Method 1.138, wherein the NRT is a nicotine patch (e.g., Nicoderm®CQ®) or oral nicotine (e.g., oral nicotine delivered by tablet or capsule) (e.g., the NRT is a nicotine patch) (e.g., the NRT is oral nicotine (e.g., oral nicotine delivered by tablet or capsule)).
1.140 Method 1.138 or 1.139, wherein the method comprises administering a subthreshold amount of the NRT (e.g., a nicotine patch, e.g., for a human that smokes more than 10 cigarettes per day, the nicotine patch starts with step 2 (i.e., one 14 mg nicotine patch per day) rather than step 1 (i.e., one 21 mg nicotine patch per day), so the human uses one 14 mg nicotine patch per day for weeks 1-6 and one 7 mg nicotine patch per day for weeks 7-8 or, e.g., the human uses one 7 mg nicotine patch per day for 8 weeks).
1.141 Any of Method 1-6, 8-15, or 1.1-1.140, wherein the human has co-morbid depression.
1.142 Any of Method 1-6, 8-15, or 1.1-1.141, wherein the human has co-morbid alcohol dependence and/or a serious mental illness with a high rate of smoking including schizophrenia or schizoaffective disorder.
1.143 Any of Method 7 or 1.1-1.10, wherein the method comprises administering 75 mg to 200 mg of (1R,5S)-1-(3,4-dichlorophenyl)-3-azabicyclo[3.1.0]hexane, in free or pharmaceutically acceptable salt form, per day, for example, comprising administering 100 mg to 200 mg of (1R,5S)-1-(3,4-dichlorophenyl)-3-azabicyclo[3.1.0]hexane, in free or pharmaceutically acceptable salt form, per day, for example, comprising administering 150 mg of (1R,5S)-1-(3,4-dichlorophenyl)-3-azabicyclo[3.1.0]hexane, in free or pharmaceutically acceptable salt form, per day (e.g., by administering 75 mg twice per day, e.g., by administering three 25 mg tablets in the morning and three 25 mg tablets in the afternoon). Any of Method 7, 1.1-1.10, or the preceding, wherein the human is an intermediate, normal, or fast nicotine metabolizer. For instance, wherein the intermediate nicotine metabolizer is heterozygous with one decreased activity CYP2A6 allele and one active CYP2A6 allele (for instance, the human has an NMR indicative of an intermediate nicotine metabolizer). For instance, wherein the normal nicotine metabolizer has two active CYP2A6 alleles (for instance, the human has an NMR indicative of a normal nicotine metabolizer). For instance, wherein the fast nicotine metabolizer has a CYP2A6 allele that produces a phenotype that increases rate of metabolism of nicotine compared to wildtype CYP2A6 (for instance, the human has an NMR indicative of a fast nicotine metabolizer). For instance, wherein the fast nicotine metabolizer has greater than two active CYP2A6 alleles (for instance, the human has an NMR indicative of a fast nicotine metabolizer).
1.144 Any of Method 7, 1.1-1.10, or 1.143, wherein the method comprises administering 50 mg to 100 mg of (1R,5S)-1-(3,4-dichlorophenyl)-3-azabicyclo[3.1.0]hexane, in free or pharmaceutically acceptable salt form, twice daily, for example, comprising administering 75 mg of (1R,5S)-1-(3,4-dichlorophenyl)-3-azabicyclo[3.1.0]hexane, in free or pharmaceutically acceptable salt form, twice daily (e.g., by administering three 25 mg tablets in the morning and three 25 mg tablets in the afternoon).
1.145 Any of Methods 1-3, 5-15, 1.1-1.26, 1.29-1.37, 1.39-1.48, 1.52-1.99, 1.101, 1.102, 1.105-1.114, 1.117, 1.118, 1.121-1.130, 1.132, 1.133, or 1.141-1.144, wherein the human has an NMR (e.g., 3HC/COT or total 3-HCOT/total COT or total 3-HCOT/COT measured in, e.g., plasma or saliva or urine) in the upper three quartiles of a measured population (e.g., the human is an intermediate, normal, or fast nicotine metabolizer). For instance, wherein the intermediate nicotine metabolizer has an NMR (e.g., 3HC/COT or total 3-HCOT/total COT or total 3-HCOT/COT measured in, e.g., plasma or saliva or urine) in the second quartile of a measured population. For instance, wherein the normal nicotine metabolizer has an NMR (e.g., 3HC/COT or total 3-HCOT/total COT or total 3-HCOT/COT measured in, e.g., plasma or saliva or urine) in the third quartile of a measured population. For instance, wherein the fast nicotine metabolizer has an NMR (e.g., 3HC/COT or total 3-HCOT/total COT or total 3-HCOT/COT measured in, e.g., plasma or saliva or urine) in the highest quartile of a measured population. Any of Methods 1-3, 5-15, 1.1-1.26, 1.29-1.37, 1.39-1.48, 1.52-1.99, 1.101, 1.102, 1.105-1.114, 1.117, 1.118, 1.121-1.130, 1.132, 1.133, 1.141-1.144, or the preceding, wherein the human has an NMR (e.g., 3HC/COT or total 3-HCOT/total COT or total 3-HCOT/COT measured in, e.g., saliva) of ≥0.18, e.g., ≥0.2, e.g., ≥0.3 (e.g., the human is an intermediate, normal, or fast nicotine metabolizer).
1.146 Any of Methods 1-3, 5-15, 1.1-1.26, 1.29-1.37, 1.39-1.48, 1.52-1.99, 1.101, 1.102, 1.105-1.114, 1.117, 1.118, 1.121-1.130, 1.132, 1.133, or 1.141-1.145, wherein the human has an NMR (e.g., 3HC/COT or total 3-HCOT/total COT or total 3-HCOT/COT measured in, e.g., plasma) of ≥0.2 (e.g., ≥0.2), e.g., ≥0.23, e.g., ≥0.25, e.g., ≥0.26, e.g., ≥0.29, e.g., ≥0.3, e.g., ≥0.35, e.g., ≥0.4, e.g., ≥0.41, e.g., ≥0.45, e.g., ≥0.5, e.g., ≥0.7, e.g., ≥0.9 (e.g., the human is an intermediate, normal, or fast nicotine metabolizer).
1.147 Any of Methods 1-3, 5-15, 1.1-1.26, 1.29-1.37, 1.39-1.48, 1.52-1.99, 1.101, 1.102, 1.105-1.114, 1.117, 1.118, 1.121-1.130, 1.132, 1.133, or 1.141-1.146, wherein the human has an NMR (e.g., 3HC/COT or total 3-HCOT/total COT or total 3-HCOT/COT measured in, e.g., plasma) of ≥0.2 (e.g., ≥0.2), e.g., ≥0.23, e.g., ≥0.25, e.g., ≥0.26, e.g., ≥0.29, e.g., ≥0.3 (e.g., the human is an intermediate, normal, or fast nicotine metabolizer).
1.148 Any of Methods 1-3, 5-15, 1.1-1.26, 1.29-1.37, 1.39-1.48, 1.52-1.99, 1.101, 1.102, 1.105-1.114, 1.117, 1.118, 1.121-1.130, 1.132, 1.133, or 1.141-1.147, wherein the intermediate nicotine metabolizer has an NMR (e.g., 3HC/COT or total 3-HCOT/total COT or total 3-HCOT/COT measured in, e.g., plasma) of ≥0.2 (e.g., ≥0.2), e.g., ≥0.23, e.g., ≥0.25, e.g., ≥0.26, e.g., ≥0.29 (e.g., 0.29), e.g., ≥0.3 or ≥0.2 (e.g., ≥0.2) to ≤0.4, e.g., ≥0.2 (e.g., ≥0.2) to ≤0.35, e.g., ≥0.2 (e.g., ≥0.2) to ≤0.3 or ≥0.3 to ≤0.4, e.g., ≥0.3 to ≤0.4.
1.149 Any of Methods 1-3, 5-15, 1.1-1.26, 1.29-1.37, 1.39-1.48, 1.52-1.99, 1.101, 1.102, 1.105-1.114, 1.117, 1.118, 1.121-1.130, 1.132, 1.133, or 1.141-1.148, wherein the intermediate nicotine metabolizer has 75% CYP2A6 activity of a normal nicotine metabolizer.
1.150 Any of Methods 1-3, 5-15, 1.1-1.26, 1.29-1.37, 1.39-1.48, 1.52-1.99, 1.101, 1.102, 1.105-1.114, 1.117, 1.118, 1.121-1.130, 1.132, 1.133, or 1.141-1.147, wherein the normal nicotine metabolizer has an NMR (e.g., 3HC/COT or total 3-HCOT/total COT or total 3-HCOT/COT measured in, e.g., plasma) of ≥0.3, e.g., ≥0.35, e.g., ≥0.4, e.g., ≥0.41 (e.g., 0.41) or ≥0.3 to ≤0.6 (e.g., ≤0.6), e.g., ≥0.3 to ≤0.5, e.g., ≥0.3 to ≤0.45, e.g., ≥0.3 to ≤0.4 or ≥0.4 to ≤0.5, e.g., ≥0.4 to ≤0.5.
1.151 Any of Methods 1-3, 5-15, 1.1-1.26, 1.29-1.37, 1.39-1.48, 1.52-1.99, 1.101, 1.102, 1.105-1.114, 1.117, 1.118, 1.121-1.130, 1.132, 1.133, or 1.141-1.147, wherein the fast nicotine metabolizer has an NMR (e.g., 3HC/COT or total 3-HCOT/total COT or total 3-HCOT/COT measured in, e.g., plasma) of ≥0.45, e.g., ≥0.5, e.g., ≥0.6, e.g., ≥0.7, e.g., ≥0.9 (e.g., 0.9) or ≥0.5 to ≤1, e.g., ≥0.5 to ≤0.9, e.g., ≥0.5 to ≤0.8, e.g., ≥0.5 to ≤0.7 or ≥0.6 to ≤1.
1.152 Any of Methods 1, 2, 4, 5, 5a, 7, 9, 10, 12-15, 1.1-1.10, 1.16-1.19, 1.22-1.28, 1.33-1.35, 1.38-1.40, 1.49-1.51, 1.80, 1.100, 1.102-1.104, 1.109-1.112, 1.115, 1.116, 1.118-1.120, 1.125-1.128, or 1.131-1.142, wherein the human has an NMR (e.g., 3HC/COT or total 3-HCOT/total COT or total 3-HCOT/COT measured in, e.g., plasma or saliva or urine) in the lowest quartile of a measured population (e.g., the human is a poor or slow nicotine metabolizer). Any of Methods 1, 2, 4, 5, 5a, 7, 9, 10, 12-15, 1.1-1.10, 1.16-1.19, 1.22-1.28, 1.33-1.35, 1.38-1.40, 1.49-1.51, 1.80, 1.100, 1.102-1.104, 1.109-1.112, 1.115, 1.116, 1.118-1.120, 1.125-1.128, 1.131-1.142, or the preceding, wherein the human has an NMR (e.g., 3HC/COT or total 3-HCOT/total COT or total 3-HCOT/COT measured in, e.g., saliva) of ≤0.3, e.g., ≤0.2, e.g., ≤0.18 (e.g., the human is a poor or slow nicotine metabolizer).
1.153 Any of Methods 1, 2, 4, 5, 5a, 7, 9, 10, 12-15, 1.1-1.10, 1.16-1.19, 1.22-1.28, 1.33-1.35, 1.38-1.40, 1.49-1.51, 1.80, 1.100, 1.102-1.104, 1.109-1.112, 1.115, 1.116, 1.118-1.120, 1.125-1.128, 1.131-1.142, or 1.152, wherein the human has an NMR (e.g., 3HC/COT or total 3-HCOT/total COT or total 3-HCOT/COT measured in, e.g., plasma) of ≤0.31, e.g., ≤0.3, e.g., ≤0.26, e.g., ≤0.23, e.g., ≤0.2, e.g., ≤0.17 (e.g., 0.17) (e.g., the human is a poor or slow nicotine metabolizer).
1.154 Any of Methods 1, 2, 4, 5, 5a, 7, 9, 10, 12-15, 1.1-1.10, 1.16-1.19, 1.22-1.28, 1.33-1.35, 1.38-1.40, 1.49-1.51, 1.80, 1.100, 1.102-1.104, 1.109-1.112, 1.115, 1.116, 1.118-1.120, 1.125-1.128, 1.131-1.142, 1.152, or 1.153, wherein the human has ≤50% CYP2A6 activity of a normal nicotine metabolizer.
1.155 Any of Methods 1, 2, 4, 5, 5a, 7, 9, 10, 12-15, 1.1-1.10, 1.16-1.19, 1.22-1.28, 1.33-1.35, 1.38-1.40, 1.49-1.51, 1.80, 1.100, 1.102-1.104, 1.109-1.112, 1.115, 1.116, 1.118-1.120, 1.125-1.128, 1.131-1.142, or 1.152-1.154, wherein the poor nicotine metabolizer has an NMR (e.g., 3HC/COT or total 3-HCOT/total COT or total 3-HCOT/COT measured in, e.g., plasma) of ≤0.2, e.g., ≤0.17.
1.156 Any of Methods 1, 2, 4, 5, 5a, 7, 9, 10, 12-15, 1.1-1.10, 1.16-1.19, 1.22-1.28, 1.33-1.35, 1.38-1.40, 1.49-1.51, 1.80, 1.100, 1.102-1.104, 1.109-1.112, 1.115, 1.116, 1.118-1.120, 1.125-1.128, 1.131-1.142, or 1.152-1.154, wherein the slow nicotine metabolizer has an NMR (e.g., 3HC/COT or total 3-HCOT/total COT or total 3-HCOT/COT measured in, e.g., plasma) of ≤0.31, e.g., ≤0.3, e.g., ≤0.26, e.g., ≤0.23, e.g., ≤0.2, e.g., ≤0.17 (e.g., 0.17).
1.157 Any of Method 1-15 or 1.1-1.156, wherein the human is female.
1.158 Any of Method 1-6, 8-15, or 1.1-1.157, wherein the human has previously been refractory to at least one (e.g., one or two or more) adequate prior course of approved treatment for nicotine dependency and nicotine withdrawal effects (e.g., wherein the human has previously been refractory to at least one adequate prior course of approved treatment for smoking cessation (e.g., an NRT)).
1.159 Any of Method 1-6, 8-15, or 1.1-1.158, wherein the human has relapsed after at least one (e.g., one or two or more) adequate prior course of successful approved treatment for nicotine dependency and nicotine withdrawal effects (e.g., wherein the human has relapsed after at least one adequate prior course of successful approved treatment for smoking cessation (e.g., an NRT)) (e.g., wherein the human has previously been refractory, e.g., wherein the human has relapsed).
1.160 Method 1.158 or 1.159, wherein the human is refractory or relapsed because of one or more nicotine withdrawal effects (e.g., one or more of quit-related changes in mood (e.g., depression and/or anxiety), anhedonia, cue-associated impulsive loss of control, and weight change (e.g., quit-related depression and/or quit-related anxiety and/or anhedonia and/or cue-associated impulsive loss of control and/or weight change and/or weight gain)).
1.161 Any of Methods 1-6, 8-15, or 1.1-1.160, wherein the one or more withdrawal effects are one or more of quit-related changes in mood (e.g., depression and/or anxiety), anhedonia, cue-associated impulsive loss of control, and weight change (e.g., quit-related depression and/or quit-related anxiety and/or anhedonia and/or cue-associated impulsive loss of control and/or weight change and/or weight gain).
1.162 Any of Methods 1-6, 8-15, or 1.1-1.161, wherein the human further receives smoking cessation counseling.

Also provided is use of (1R,5S)-1-(3,4-dichlorophenyl)-3-azabicyclo[3.1.0]hexane, in free or pharmaceutically acceptable salt form, in the manufacture of a medicament for use in any of Method 1-15 or 1.1-1.162.

Also provided is a pharmaceutical composition comprising (1R,5S)-1-(3,4-dichlorophenyl)-3-azabicyclo[3.1.0]hexane, in free or pharmaceutically acceptable salt form, in combination with a pharmaceutically acceptable diluent or carrier for use in any of Method 1-15 or 1.1-1.162.

Also provided is use of (1R,5S)-1-(3,4-dichlorophenyl)-3-azabicyclo[3.1.0]hexane, in free or pharmaceutically acceptable salt form, in any of Method 1-15 or 1.1-1.162.

In some embodiments described herein, the intermediate, normal, or fast nicotine metabolizer has an NMR (e.g., 3HC/COT or total 3-HCOT/total COT or total 3-HCOT/COT measured in, e.g., plasma or saliva or urine) in the upper three quartiles of a measured population.

In some embodiments described herein, the intermediate nicotine metabolizer has an NMR (e.g., 3HC/COT or total 3-HCOT/total COT or total 3-HCOT/COT measured in, e.g., plasma or saliva or urine) in the second quartile of a measured population.

In some embodiments described herein, the normal nicotine metabolizer has an NMR (e.g., 3HC/COT or total 3-HCOT/total COT or total 3-HCOT/COT measured in, e.g., plasma or saliva or urine) in the third quartile of a measured population.

In some embodiments described herein, the fast nicotine metabolizer has an NMR (e.g., 3HC/COT or total 3-HCOT/total COT or total 3-HCOT/COT measured in, e.g., plasma or saliva or urine) in the highest quartile of a measured population.

In some embodiments described herein, the intermediate, normal, or fast nicotine metabolizer has an NMR (e.g., 3HC/COT or total 3-HCOT/total COT or total 3-HCOT/COT measured in, e.g., saliva) of ≥0.18, e.g., ≥0.2, e.g., ≥0.3

In some embodiments described herein, the intermediate, normal, or fast nicotine metabolizer has an NMR (e.g., 3HC/COT or total 3-HCOT/total COT or total 3-HCOT/COT measured in, e.g., plasma) of ≥0.2 (e.g., ≥0.2), e.g., ≥0.23, e.g., ≥0.25, e.g., ≥0.26, e.g., ≥0.29, e.g., ≥0.3, e.g., ≥0.35, e.g., ≥0.4, e.g., ≥0.41, e.g., ≥0.45, e.g., ≥0.5, e.g., ≥0.7, e.g., ≥0.9.

In some embodiments described herein, the intermediate nicotine metabolizer has an NMR (e.g., 3HC/COT or total 3-HCOT/total COT or total 3-HCOT/COT measured in, e.g., plasma) of ≥0.2 (e.g., ≥0.2), e.g., ≥0.23, e.g., ≥0.25, e.g., ≥0.26, e.g., ≥0.29 (e.g., 0.29), e.g., ≥0.3 or ≥0.2 (e.g., ≥0.2) to ≤0.4, e.g., ≥0.2 (e.g., ≥0.2) to ≤0.35, e.g., ≥0.2 (e.g., ≥0.2) to ≤0.3 or ≥0.3 to ≤0.4, e.g., ≥0.3 to ≤0.4.

In some embodiments, the intermediate nicotine metabolizer has 75% CYP2A6 activity of a normal nicotine metabolizer.

In some embodiments described herein, the normal nicotine metabolizer has an NMR (e.g., 3HC/COT or total 3-HCOT/total COT or total 3-HCOT/COT measured in, e.g., plasma) of ≥0.3, e.g., ≥0.35, e.g., ≥0.4, e.g., ≥0.41 (e.g., 0.41) or ≥0.3 to ≤0.6, e.g., ≥0.3 to ≤0.5, e.g., ≥0.3 to ≤0.45, e.g., ≥0.3 to ≤0.4 or ≥0.4 to ≤0.5, e.g., ≥0.4 to ≤0.5.

In some embodiments described herein, the fast nicotine metabolizer has an NMR (e.g., 3HC/COT or total 3-HCOT/total COT or total 3-HCOT/COT measured in, e.g., plasma) of ≥0.45, e.g., ≥0.5, e.g., ≥0.6, e.g., ≥0.7, e.g., ≥0.9 (e.g., 0.9) or ≥0.5 to ≤1, e.g., ≥0.5 to ≤0.9, e.g., ≥0.5 to ≤0.8, e.g., ≥0.5 to ≤0.7 or ≥0.6 to ≤1.

In some embodiments described herein, the poor or slow nicotine metabolizer has an NMR (e.g., 3HC/COT or total 3-HCOT/total COT or total 3-HCOT/COT measured in, e.g., plasma or saliva or urine) in the lowest quartile of a measured population.

In some embodiments described herein, the poor or slow nicotine metabolizer has an NMR (e.g., 3HC/COT or total 3-HCOT/total COT or total 3-HCOT/COT measured in, e.g., saliva) of ≤0.3, e.g., ≤0.2, e.g., ≤0.18.

In some embodiments described herein, the poor or slow nicotine metabolizer has an NMR (e.g., 3HC/COT or total 3-HCOT/total COT or total 3-HCOT/COT measured in, e.g., plasma) of ≤0.31, e.g., ≤0.3, e.g., ≤0.26, e.g., ≤0.23, e.g., ≤0.2, e.g., ≤0.17 (e.g., 0.17).

In some embodiments described herein, the poor or slow nicotine metabolizer has ≥50% CYP2A6 activity of a normal nicotine metabolizer.

In some embodiments described herein, the poor nicotine metabolizer has an NMR (e.g., 3HC/COT or total 3-HCOT/total COT or total 3-HCOT/COT measured in, e.g., plasma) of ≤0.2, e.g., ≤0.17.

In some embodiments described herein, the slow nicotine metabolizer has an NMR (e.g., 3HC/COT or total 3-HCOT/total COT or total 3-HCOT/COT measured in, e.g., plasma) of ≤0.31, e.g., ≤0.3, e.g., ≤0.26, e.g., ≤0.23, e.g., ≤0.2, e.g., ≤0.17 (e.g., 0.17).

Dosages employed in practicing the present disclosure will vary depending, for example, on the mode of administration and the therapy desired. A daily dosage of (1R,5S)-1-(3,4-dichlorophenyl)-3-azabicyclo[3.1.0]hexane, in free or pharmaceutically acceptable salt form, for oral administration to an intermediate, a normal, or a fast nicotine metabolizer may be in the range of from 75 to 200 mg per day, for example, 100 mg to 200 mg per day, for example, 150 mg per day, conveniently administered once or in divided doses 2 to 6 times daily, optionally in sustained release form. A daily dosage of (1R,5S)-1-(3,4-dichlorophenyl)-3-azabicyclo[3.1.0]hexane, in free or pharmaceutically acceptable salt form, for oral administration to a poor or slow nicotine metabolizer may be in the range of from 25 to 75 mg per day conveniently administered once or in divided doses daily, optionally in sustained release form.

Unit dosage forms for oral administration thus may comprise from, for example, 25 mg to 200 mg, for example, from 25 mg or 50 mg or 75 mg or 100 mg or 150 mg to 200 mg, for example, from 100 mg to 150 mg, of (1R,5S)-1-(3,4-dichlorophenyl)-3-azabicyclo[3.1.0]hexane, in free or pharmaceutically acceptable salt form, together with a pharmaceutically acceptable diluent or carrier therefor.

A method of administration of the dose of the present disclosure is not particularly limited. (1R,5S)-1-(3,4-dichlorophenyl)-3-azabicyclo[3.1.0]hexane, in free or pharmaceutically acceptable salt form, may be administered by any suitable route, including orally, parenterally, transdermally, inhalation, slow release, controlled release, although various other known delivery routes, devices and methods can likewise be employed. In some embodiments, provided is an oral sustained release pharmaceutical composition comprising (1R,5S)-1-(3,4-dichlorophenyl)-3-azabicyclo[3.1.0]hexane, in free or pharmaceutically acceptable salt form. In some embodiments, provided is an oral immediate release pharmaceutical composition comprising (1R,5S)-1-(3,4-dichlorophenyl)-3-azabicyclo[3.1.0]hexane, in free or pharmaceutically acceptable salt form.

Pharmaceutical compositions comprising (1R,5S)-1-(3,4-dichlorophenyl)-3-azabicyclo[3.1.0]hexane, in free or pharmaceutically acceptable salt form, may be prepared using conventional diluents or excipients and techniques known in the galenic art. Thus, oral dosage forms may include tablets, capsules, solutions, suspensions, and the like.

### EXAMPLE

### Example 1

### Abbreviations

Conc Concentration
CYP Cytochrome P450
EDTA Ethylenediaminetetraacetic acid
EMA European Medicines Agency
FDA Food and Drug Administration
HPLC High performance liquid chromatography
IC₅₀ Concentration of an inhibitor that causes a 50% decrease in enzyme activity
Kᵢ Intrinsic inhibition constant (in units of inhibitor concentration) that defines the affinity of a reversible inhibitor for an enzyme
Kₘ Substrate concentration supporting half the maximum rate of an enzyme-catalyzed reaction
LC Liquid chromatography
LC-MS/MS Liquid chromatography tandem mass spectrometry
MBI Mechanism-based inhibition; describes the formation of an inhibitory metabolite by the same enzyme that is inhibited
MDI Metabolism-dependent inhibition (or inhibitor); an increase in inhibitory potency that is observed when the test article is preincubated with human liver microsomes in the presence of NADPH
MS Mass spectrometry
n Number of replicates
NA Not applicable
NADP β-Nicotinamide adenine dinucleotide phosphate, oxidized form
NADPH β-Nicotinamide adenine dinucleotide phosphate, reduced form
NSC No solvent control
S₅₀ Substrate concentration for allosteric kinetics which supports half the maximum rate of an enzyme-catalyzed reaction
SC Solvent control
SE Standard error
TA Test article
TDI Time-dependent inhibition; an increase in inhibitory potency that is observed when the test article is preincubated with human liver microsomes in the absence of NADPH
TIC Test article interference check
TSC Test article suppression check

### SUMMARY

To evaluate amitifadine as a direct, time- and metabolism-dependent inhibitor of CYP2A6 activity, human liver microsomes from a pool of 200 individuals are incubated with marker substrate in the presence or absence of test article. To distinguish between time- and metabolism-dependent inhibition, amitifadine is preincubated with human liver microsomes for 30 min without and with an NADPH-regenerating system, respectively, prior to the incubation with the marker substrate. Known direct and metabolism-dependent inhibitors of CYP2A6 are included as positive controls.

Amitifadine directly inhibits CYP2A6 with an IC₅₀ value of 0.42 μM. There is no evidence of metabolism-dependent inhibition of CYP2A6.

### 1. Introduction

### 1.1 Evaluation of Drugs as Inhibitors of CYP Enzymes

Drugs that inhibit CYP enzymes can do so directly or indirectly. Both mechanisms of inhibition, direct and indirect, can be further subdivided. Direct inhibition can be attributable to the parent drug (or drug candidate) and involves the binding of the parent molecule to the CYP enzyme. Depending on the location of the CYP binding site, direct-acting inhibitors can be characterized as competitive, noncompetitive, uncompetitive, or mixed inhibitors. The affinity with which they inhibit enzyme activity is expressed as Kᵢ (the intrinsic inhibition constant), which has units of inhibitor concentration.

Indirect inhibition is attributable to one or more metabolites of the parent drug (or drug candidate). Indirect inhibition is often referred to as time-dependent inhibition (TDI), metabolism-dependent inhibition (MDI) and mechanism-based inhibition (MBI), but these definitions are not synonymous. Mechanism-based inhibition involves the formation of an inhibitory metabolite by the same enzyme that becomes inhibited. It represents a true case of auto-inhibition. Mechanism-based inhibitors of CYP enzymes can be further subdivided into quasi-irreversible and irreversible inhibitors.

Mechanism-based inhibition is one of two types of metabolism-dependent inhibition; the other involves the formation of an inhibitory metabolite not by the CYP enzyme that becomes inhibited but by another enzyme, such as another form of cytochrome P450 or another type of drug-metabolizing enzyme.

Metabolism-dependent inhibition is often used synonymously with time-dependent inhibition. In the majority of cases, this causes no confusion because the formation of inhibitory metabolites takes time; therefore, all metabolism-dependent inhibitors are time-dependent inhibitors. However, the converse is not true; not all time-dependent inhibitors are metabolism-dependent inhibitors. Some inhibitors have a slow on-rate; meaning they complex slowly with the enzyme they inhibit. In such cases, the observed inhibition is attributable to the parent compound, but the degree of inhibition increases over time. In this respect, they resemble metabolism-dependent inhibitors. For studies conducted with human liver microsomes, the term ‘metabolism-dependent inhibition’ is used to describe an increase in inhibitory potency observed when the test article is incubated with human liver microsomes in the presence of NADPH, and the term ‘time-dependent inhibition’ is used to describe an increase in inhibitory potency observed when the test article is incubated with human liver microsomes in the absence of NADPH.

This study is designed to evaluate the ability of amitifadine to inhibit CYP2A6 in human liver microsomes. The inhibitory potency of amitifadine is determined in vitro by measuring the activity of CYP2A6 in human liver microsomes in the presence and absence of amitifadine. The in vitro experiment is designed to measure the concentration of inhibitor (amitifadine) that causes 50% inhibition of marker substrate activity (IC₅₀ value) for direct and time- and metabolism-dependent inhibition of CYP2A6.

### 1.2 Determination of IC₅₀ Values

Amitifadine is evaluated for its ability to function as a direct, time- or metabolism-dependent inhibitor of CYP2A6 as measured by coumarin 7-hydroxylation.

### 2. Materials and Methods

### 2.1 Materials

All reagents and solvents are of analytical grade. The purified water is prepared with a Labconco WaterPro PS Polishing system or a Millipore Milli-Q Advantage A-10 system.

### 2.2 Test System

Human liver microsomes from non-transplantable, donated livers are prepared and characterized at the testing facility as outlined by Pearce, R. et al., “Effects of Freezing, Thawing, and Storing Human Liver Microsomes on Cytochrome P450 Activity,” Archives of Biochemistry and Biophysics, 1996, 331 (2), 145-169 and Parkinson, A. et al., “The Effects of Gender, Age, Ethnicity and Liver Cirrhosis on Cytochrome P450 Enzyme Activity in Human Liver Microsomes and Inducibility in Cultured Human Hepatocytes,” Toxicology and Applied Pharmacology, 2004, 199 (3), 193-209. A mixed-gender pool of 200 individual human liver microsomal samples is used for this study (Sekisui XenoTech catalog number: H2610). The kinetic constants (S₅₀ or Kₘ) used to select marker substrate concentrations and incubation conditions are determined previously.

### 2.3 Test Article

A stock solution of the test article (target concentration of 2 mM) is prepared in water, and qualitative solubility testing of the test article in the test system is conducted. An aliquot (100 μL) of the stock amitifadine solution is added to a 900-μL, mixture (pH 7.4) containing water, potassium phosphate buffer (50 mM), MgCl₂ (3 mM), EDTA (1 mM) and human liver microsomes (0.0125 mg/mL) for a total volume of 1000 μL. Visual comparison of the tube to which amitifadine is added with a control tube containing the same components without test article indicate that amitifadine is soluble in the test system at 200 μM. Since stability of the test article solutions is not known, the stock solution and dilutions to working solutions are prepared fresh on the day of use.

### 2.4 Evaluation of Amitifadine as an Inhibitor of Human CYP2A6 in Human Liver Microsomes

The ability of amitifadine to inhibit CYP2A6 is investigated with a pool of 200 individual human liver microsomal samples (Section 2.2). The final target concentration of amitifadine ranges from 0.2 to 200 μM (Table 1). The basis for the following incubation conditions is described in the following references: European Medicines Agency (2013) Guideline on the investigation of drug interactions. European Medicines Agency, London. 60 p. EMA Guideline No.: CPMP/EWP/560/95/Rev.1 Corr.; Food and Drug Administration (2012) Draft guidance for industry: Drug interaction studies—study design, data analysis, implications for dosing, and labeling recommendations, U.S. Department of Health and Human Services, Rockville, Md. 79 p.; Ogilvie, B. et al., In vitro Approaches for Studying the Inhibition of Drug-metabolizing Enzymes Responsible for the Metabolism of Drugs (Reaction Phenotyping) with Emphasis on Cytochrome P450, in Drug-Drug Interactions, 2008, 2^{nd} Ed. (Rodrigues A., Ed), pp. 231-358, Informa Healthcare USA, Inc., New York; Ogilvie, B. et al., “Glucuronidation Converts Gemfibrozil to a Potent, Metabolism-dependent Inhibitor of CYP2C8: Implications for Drug-drug Interactions,” Drug Metabolism & Disposition, 2006, 34 (1), 191-197; Pearce, R. et al., “Effects of Freezing, Thawing, and Storing Human Liver Microsomes on Cytochrome P450 Activity,” Archives of Biochemistry and Biophysics, 1996, 331 (2), 145-169; Tucker, G. et al., “Optimizing Drug Development: Strategies to Assess Drug Metabolism/Transporter Interaction Potential—Toward a Consensus,” Pharmaceutical Research, 2001, 18 (8), 1071-1080; Walsky, R. et al., “Validated Assays for Human Cytochrome P450 Activities,” Drug Metabolism & Disposition, 2004, 32 (6), 647-660.

TABLE 1
IC₅₀ determinations: Summary of assay conditions to measure microsomal CYP enzyme activity -
Direct, time-dependent and metabolism-dependent inhibition of CYP2A6 by amitifadine

| | | | | | | | Amitifadine | |
|---|---|---|---|---|---|---|---|---|
| | | Substrate | Incubation | | Incubation | Preincubation | Target | Solvent |
| | | Concentration | volume | Protein | time | time | concentrations | volume |
| Enzyme | Substrate | (μM) | (μL) | (mg/mL)^{a} | (min) | (min) | (μM) | (μL)^{b} |
| CYP2A6 | Coumarin | 0.6 | 200 | 0.0125 | 5 | 30 | 0, 0.2, 0.6, 2, 6, | 20 |
| | | | | | | | 20, 60, 200 | |

^{a}The human liver microsomal sample used for this experiment is a pool of 200 individuals catalog number: H2610).
^{b}Water is the solvent used to dissolve the test article.

Aliquots of the stock and/or working solutions of amitifadine are manually added to buffer mixtures (target pH 7.4) containing water, potassium phosphate buffer (50 mM), MgCl₂ (3 mM), EDTA (1 mM) and human liver microsomes (0.0125 mg/mL). Incubation mixtures are prepared in bulk to obviate the need for directly pipetting very small volumes (i.e., 1 μL or less). The test article is dissolved in water and added directly to incubation mixtures.

Incubations measuring residual enzyme activity are conducted at 37±2° C. in 200-4, incubation mixtures (pH 7.4) containing the buffer mixture described previously, an NADPH-regenerating system (NADP [1 mM], glucose-6-phosphate [5 mM], glucose-6-phosphate dehydrogenase [1 Unit/mL]), and a concentration of marker substrate based on internal Kₘ or S₅₀ data. A Tecan liquid handling system conducts all incubations. Duplicate aliquots of the buffer mixtures are automatically added to 96-well plates at the appropriate locations. Aliquots of a substrate working solution are added to the 96-well plates prior to initiating reactions to give the final concentrations indicated in Table 1. Marker substrate reactions are initiated by the addition of an aliquot of an NADPH-regenerating system and are automatically terminated at approximately 5 min by the addition of the appropriate internal standard (Table 2) and stop reagent, acetonitrile. The samples are centrifuged at 920×g for 10 min at 10° C. The supernatant fractions are analyzed by liquid chromatography tandem mass spectrometry (LC-MS/MS). Calibration standards are similarly prepared with the addition of authentic metabolite standard.

TABLE 2
Summary of metabolite analysis by liquid
chromatography tandem mass spectrometry

| | | Electrospray | | Internal |
|---|---|---|---|---|
| | Metabolite | ionization | Internal | standard |
| Enzyme | monitored | mode | standard | concentration^{a} |
| CYP2A6 | 7-Hydroxy- | Negative | 7-Hydroxy- | 150 ng/mL |
| | coumarin | | coumarin-d₅ | |

^{a}Value indicates the stock concentration of internal standard. This concentration is diluted 16-fold when added to the stopped incubation mixture.

To examine its ability to act as a metabolism-dependent inhibitor of CYP2A6, amitifadine (at the same concentrations used to evaluate direct inhibition) is preincubated at 37±2° C., in duplicate, with human liver microsomes and an NADPH-regenerating system for approximately 30 min. This preincubation allows for the generation of intermediates that may inhibit human CYP2A6. The preincubations are initiated by the addition of an aliquot of an NADPH-regenerating system. To examine its ability to act as a time-dependent inhibitor of CYP2A6, additional duplicate samples at all test article concentrations are preincubated for 30 min in the presence of pooled human liver microsomes, but in the absence of NADPH. This preincubation allows assessment of whether any potential increase in inhibition is dependent upon NADPH-dependent metabolism (e.g., potentially CYP-mediated). Following the 30-min preincubation period, NADPH and/or marker substrate is added, and the incubations are continued as described previously to measure residual CYP2A6 activity. Incubations containing no test article (0 μM; solvent control), and incubations that contain test article but are not preincubated serve as negative controls.

### 2.5 Analytical Methods

Metabolite analysis is performed with an LC-MS/MS method. The procedure used for the analysis follows the applicable LC-MS/MS analytical method SOP and is summarized in Table 2. The MS equipment is an appropriate AB SCIEX mass spectrometer with Shimadzu LC pumps and autosampler systems (Table 3).

TABLE 3

| | Metabolite | Mass | HPLC |
|---|---|---|---|
| Enzyme | monitored | spectrometer | column^{a} |
| CYP2A6 | 7-Hydroxy- | API 3000 triple | Waters Atlantis dC18 |
| | coumarin | quadrupole | (5 μm, 100 × 2.1 mm) |

^{a}All HPLC columns are preceded by a Phenomenex Luna C-8 guard column (4.0 mm × 2.0 mm).

An authentic metabolite standard is used to quantify the metabolite, and deuterated metabolite is used as an internal standard. Zero-time incubations serve as blanks.

The metabolite is quantified by reference to a standard calibration curve generated using the simplest appropriate weighting and regression algorithm. The regression fit is based on the peak area ratio of the analyte to internal standard calculated from calibration standard samples. Stock standard solutions and working solutions are prepared according to the custom Tecan script EVO Std-QC Spiking Solution Prep. Chromatographic peaks are integrated with Analyst Instrument Control and Data Processing Software (AB SCIEX, version 1.6.1).

### 2.6 Statistical Tests and Data Processing

Data are processed with a LIMS (includes Galileo version 3.3, Thermo Fisher Scientific Inc. and reporting tool, Crystal Reports 2013, SAP). When inhibition is observed, the data are processed for the determination of IC₅₀ values by non-linear regression and displayed on an appropriate plot. This LIMS utilizes the Levenberg-Marquardt algorithm to perform non-linear regression fitting of the data to the following 4-parameter sigmoidal-logistic IC₅₀ equation:

$$Y = Min + \frac{( Max - Min )}{( 1 + {( Conc / {IC}_{50} )}^{slope} )}$$

As percent of control values are utilized, the minimum value (Min) is set to zero, and the maximum value (Max) is set to 100 (or other values, as appropriate). This software has been verified for its ability to calculate an IC₅₀ value only when it lies within the concentration range of inhibitor studied.

### 2.7 Assay Controls

### 2.7.1 TSC and TIC Samples

Additional incubations (with and without preincubation) that contain the highest concentration of amitifadine, but no probe substrate, are included to assess for analytical interference by amitifadine and/or possible metabolite(s) in the method used to determine CYP2A6 activity.

### 2.7.2 Positive Controls

Known direct and metabolism-dependent inhibitors of CYP2A6 are included as positive controls. The direct inhibitor is incubated for the normal incubation time and with the normal protein concentration in the presence of the marker substrate. The metabolism-dependent inhibitor is preincubated with human liver microsomes for zero and 30 min followed by incubation with marker substrate for the normal incubation time and with the normal protein concentration as described in Section 2.4.

### 3. Results and Discussion

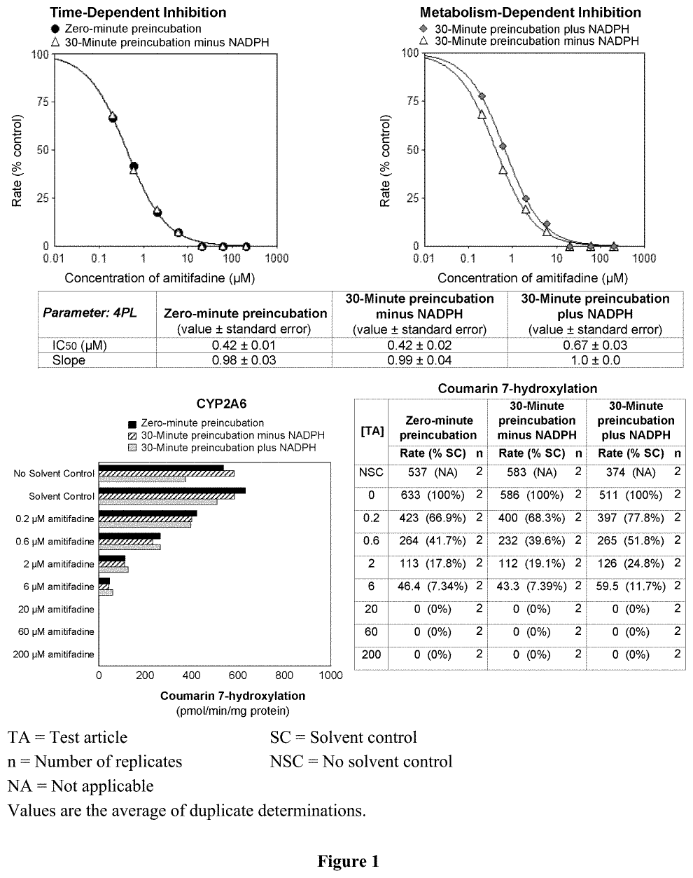

The evaluation of amitifadine as a direct, time- and metabolism-dependent inhibitor of human CYP2A6 is summarized in Table 4 and FIG. 1.

TABLE 4
Summary of results: In vitro evaluation of amitifadine as
an inhibitor of human CYP2A6 in human liver microsomes

| | | | | | | Metabolism- | | |
|---|---|---|---|---|---|---|---|---|
| | | | | | | dependent | | |
| | | | | Time-dependent | | inhibition^{c} | | |
| | | Direct inhibition | | inhibition | | 30-min | | |
| | | Zero-min | | 30-min preincubation | | preincubation | | |
| | | preincubation | | without NADPH | | with NADPH | | |
| | | | Max. | | Max. | | Max. | Type of |
| | | IC₅₀ ± SE | inhibition | IC₅₀ ± SE | inhibition | IC₅₀ ± SE | inhibition | inhibition |
| Enzyme | Substrate | (μM)^{a} | (%)^{b} | (μM)^{a} | (%)^{b} | (μM)^{a} | (%)^{b} | observed |
| CYP2A6 | Coumarin | 0.42 ± 0.01 | 100 | 0.42 ± 0.02 | 100 | 0.67 ± 0.03 | 100 | Direct |

SE = Standard error
^{a}Average data (i.e., percent of control activity) obtained from duplicate samples for each test article concentration are used to calculate IC₅₀ values.
^{b}Maximum inhibition observed (%) is calculated with the following formula: Maximum inhibition observed (%) = 100% − the minimum percent solvent control for any test article concentration.
^{c}Metabolism-dependent inhibition is determined by comparison of IC₅₀ values both with and without preincubation and with and without NADPH-regenerating system present in the preincubation, by comparison of the observed inhibition (%) for all preincubation conditions and by visual inspection of the IC₅₀ plots.

### 3.1 Evaluation of Amitifadine as an Inhibitor of Human CYP2A6 in Human Liver Microsomes: Determination of IC₅₀ Values

Amitifadine directly inhibits CYP2A6 with an IC₅₀ value of 0.42 μM. There is no evidence of metabolism-dependent inhibition of CYP2A6.

Experiments described in Section 2.4 involve preincubating human liver microsomes in the presence of an NADPH-regenerating system but in the absence of marker substrate. When such incubations are carried out, some loss in activity of the enzyme tested is observed regardless of the presence of amitifadine. This loss in enzyme activity is attributed to inactivation of CYP2A6 (e.g., by reactive oxygen species, Zangar R. et al., “Mechanisms that Regulate Production of Reactive Oxygen Species by Cytochrome P450,” Toxicology and Applied Pharmacology, 2004, 199 (3), 316-331). It does not have a negative impact on the data interpretations or conclusions.

### 3.2 Assay Controls

### 3.2.1 TSC and TIC Samples

In all cases, the test article interference and test article suppression check samples show that the addition of amitifadine to the test system has no impact on the analytical method. The data for these samples are not reported.

### 3.2.2 Positive Controls for IC₅₀ Determinations

The positive control inhibitors for direct inhibition and metabolism-dependent inhibition (Table 5) inhibit CYP2A6 enzyme activity as expected.

TABLE 5
Positive control inhibition data for IC₅₀ determination

| | | | | | | Increase in |
|---|---|---|---|---|---|---|
| | | | | | 30-min | inhibition |
| | | | | Zero-min | preincubation | after |
| | | Type of | | preincubation | plus NADPH) | preincubation |
| Enzyme | Substrate | inhibitor | Inhibitor | (% inhibition) | (% inhibition) | (%) |
| CYP2A6 | Coumarin | Direct | 300 μM Nicotine | 82.5 | NA | NA |
| | | MDI | 0.05 μM 8- | 3.06 | 74.9 | 71.8 |
| | | | Methoxypsoralen | | | |

MDI = Metabolism-dependent inhibitor
NA = Not applicable

### 4. Conclusions

Amitifadine directly inhibits CYP2A6 with an IC₅₀ value of 0.42 μM.

There is no evidence of metabolism-dependent inhibition of CYP2A6.

### Example 2

Individuals who smoke daily and who express a desire to quitting smoking, and who are not currently using e-cigarettes or other tobacco products (within the past 30 days) will be recruited. Interested potential subjects will be given a brief description of the study and will be asked questions to assess eligibility and interest, which includes being in the “preparation” stage of smoking cessation (Prochaska, J. et al., “In Search of How People Change. Applications to Addictive Behaviours,” American Pscyhologist, 1992, 47, 1102-1114), i.e. ready to quit smoking within the next 30 days. Eligible subjects interested in participating will be scheduled for the screening history and physical examination. A physical examination and an ECG will be performed on eligible subjects. Participants will be screened by medical history, physical examination, blood tests and urinalysis to rule out major medical conditions. The initial target quit date for all the subjects will be established at screening.

This study will a) evaluate the efficacy of amitifadine in terms of promoting smoking abstinence and reducing smoking withdrawal symptoms, including negative affect accompanying smoking abstinence; and b) assess tolerability and adverse side effects. The study design is a randomized, placebo-controlled parallel arm clinical trial that will include 200 male and female smokers (50/group). Participants will be randomly assigned in a 2×2 factorial design to four treatment groups that receive either: 1) amitifadine 150 mg/day+21 mg/24 h nicotine patch (“amitifadine+NRT” condition); 2) placebo amitifadine+21 mg/24 h nicotine patch (“NRT alone” condition); 3) amitifadine 150 mg/day+placebo nicotine patches (“amitfadine alone” condition); or 4) placebo amitifadine+placebo nicotine patch (“placebo” condition). All participants, regardless of group allocation, will be asked to take three capsules twice daily, once in the morning and once in the early evening.

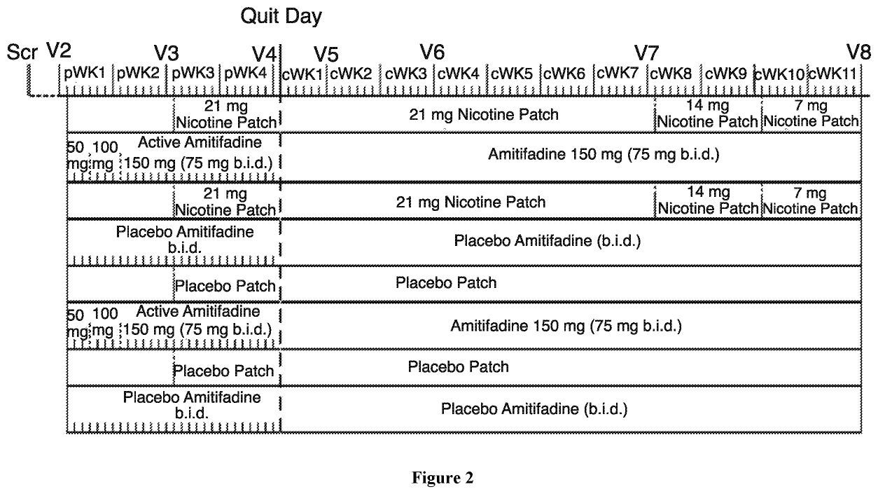

Dosing will begin 4 weeks before a target quit-smoking date, and will continue for 11 weeks post-quit (the post-quit date duration treatment for other FDA-approved pharmacotherapies bupropion and varenicline). Active medications and placebo will be provided as follows (see also FIG. 2):
Amitifadine 150 mg/day dosing: First 3 days of week 1: AM—two 25 mg capsules and one placebo capsule; PM—three placebo capsules.
Next 4 days of week 1: AM—two 25 mg capsules and one placebo capsule; PM—two 25 mg capsules and one placebo capsule.
Rest of treatment: AM—three 25 mg capsules; PM—three 25 mg capsules.
Nicotine patch
Nicotine patch dosing will use the standard 21 mg/24 h patch (Nicoderm). FDA-approved labeling allows continuation of patch dosing with concurrent smoking, which will occur during the 2 weeks before the target quit date.

In the event of significant adverse reactions (e.g., nausea, vomiting), a dose reduction will be implemented whereby the twice-daily capsule dose is reduced from three (twice a day) to two to one, or discontinued altogether, and the patch dose will be concomitantly reduced from 21 mg to 14 mg to 7 mg, or discontinued altogether. Sleep disturbances will initially be handled by instruction subjects to remove patches at bedtime. These procedures will maintain the double blind as to condition assignment.

Treatment related side effects will also be assessed by open-ended as well as targeted questionnaires. These include diarrhea, nausea, rash, abdominal pain, and sexual function assessed by the Change in Sexual Functioning Questionnaire.

At baseline, demographics will also be assessed for covariate analysis of study results. In addition to demographic variables of age, sex, race and ethnicity, assess smoking history (e.g., number of prior quit attempts) and socioeconomic (SES) variables (e.g., education, income) will be assessed. During each session, expired air CO will be measured along with blood pressure, heart rate and body weight. Blood samples will be collected at each session for analysis of amitifadine levels. Smoking and medication diaries and any other take-home forms will be collected and reviewed. Participants will also complete questionnaires rating (for that day) craving, mood, withdrawal symptoms, side effects, and subjective effects of smoking. Information on alcohol and caffeine consumption since the last visit will also be gathered. Blood for serum chemistries and assay of amitifadine and cotinine levels, complete blood count (CBC) and urine for urinalysis will also be collected at each study session. Subjects will also be given the clinical trial materials (CTM), including active or matching placebo capsules that they will need until their next scheduled session, along with three extra days' worth of CTM to allow for flexibility in case sessions need to be rescheduled. Any unused medications will be returned for proper disposal and compliance monitoring. To enhance and assess adherence, subjects will receive twice daily text messages reminding and inquiring about when each dose is taken. A modest financial incentive ($5/day) will be provided to participants for responding to these messages and reporting dosing times.

Pre-quit study session procedures: At baseline, and at the end of week 2 and week 4, participants will be assessed of the early efficacy measures described under Aim 2 below. The quit-smoking date will be scheduled for the day after the session held at the end of study week 4.

Post-quit study session procedures: Sessions conducted after the quit-smoking date will be conducted at 2-week intervals before the quit-date and 4-week intervals until the end of week 11 post-quit. Efficacy and tolerability measures pertaining to Aim 1 (see below) will be collected. Participants who are abstinent from smoking at the end of treatment will be followed up at 6 months after the quit date. Commonly 6 months or 1 year is chosen as a follow-up interval, but given that studies show that approximately 90% of relapses that occur within a year occur within the first 6 months (Blondal, T. et al., “Nicotine Nasal Spray with Nicotine Patch for Smoking Cessation: Randomised Trial with Six Year Follow Up,” BMJ, 1999, 30 (318), 285-288), a 6-month follow-up will be used. Due to ethical considerations related to the placebo condition, which will not provide the standard of care with respect to pharmacotherapy, any participant who withdraws from the study or does not achieve smoking abstinence will be offered a free course of NRT (patches, gum, lozenge) to assist them in quitting smoking.

Additionally, at all sessions, brief (<10 min) smoking cessation counseling will be provided, consistent with the Agency for Healthcare Research and Quality (AHRQ) guidelines. This counseling will address issues relevant to quit-smoking success, including maintaining the motivation to remain abstinent, dealing with nicotine withdrawal symptoms, avoiding high-risk environments that may provoke relapse, and managing weight gain. Participants will not receive intensive behavioral treatment, for two reasons: 1) hope to mimic real-world clinics where minimal behavioral support is available; and 2) inter-subject (or inter-group) variations in the intensity of behavioral treatment could confound an assessment of the effects of pharmacotherapy.

Compensation of participants: In addition to the above-mentioned $5/day compensation for reporting medication adherence, there will be a payment of $50 per laboratory session attended. Subjects claiming to be abstinent from smoking at the 6 month follow-up after the target quit date will be asked to return for an expired air CO breath test, and will receive $50 for attending this session.

Outcomes include the following: Smoking abstinence at each timepoint (primary outcome will be abstinence from weeks 8-11 post-quit date). Secondary outcome measures will include: 1) craving and nicotine withdrawal symptoms; 2) ratings of the rewarding/aversive effects of smoking; 3) smoking behavior and nicotine dependence; 4) depressed mood; including anhedonia; 5) body weight; 6) sexual functioning; 7) impulsiveness; and 8) serum amitifadine levels.

Aim 1: To evaluate the efficacy and tolerability of amitifadine alone, and in combination with NRT, for smoking cessation.

Aim 2: To assess effects of amitifadine alone, and in combination with NRT, on smoking reduction prior to the quit date, and correlate this pre-quit smoking reduction with abstinence outcomes. Research has shown that abstinence at the end of treatment is strongly predicted by the extent to which smokers spontaneously reduce ad libitum smoking before the quit-smoking date. This early marker of subsequent success can ultimately be useful in clinical practice by helping to guide adaptive changes in treatment before a potential relapse occurs. We will assess the effect of amitifadine and combination amitifadine+NRT on smoking reduction, indexed by smoking diaries and biochemical measures (expired air carbon monoxide and cotinine levels), and we will correlate these reductions with abstinence assessed at the end of treatment.

Exploratory Aim 3: To identify additional early predictors of treatment outcome. In addition to pre-quit reductions in smoking, other variables will be assessed before the quit-smoking date, and correlated with end-of-treatment abstinence. Key objective measures include serum amitifadine levels and nicotine metabolite ratio. Subjective ratings of the rewarding effects of smoking, craving and other nicotine withdrawal symptoms, will be assessed using standardized questionnaires. Additionally, we will assess the predictive value of demographic measures, including body weight, gender, age, race, as well as smoking history variables, including baseline cigarette consumption, level of nicotine dependence, and number of prior quit attempts.

### Outcomes:

The primary abstinence outcome will be abstinence from weeks 8-11 post-quit date. This will be defined by a self-report of no cigarette smoking (not even a puff) over the 4-week interval from week 8 to week 11 post-quit. Self-reported abstinence will be confirmed by an expired air CO reading of <7 ppm.

A secondary smoking abstinence outcome will be point (7-day) abstinence at 6 months post-quit, for subjects who have not relapsed prior to the end of treatment. Those who have relapsed or dropped out prior to week 11 will be offered free NRT, which compromises the assessment of treatment effects at follow-up. However, the main goal of the 6-month follow-up is to assess the persistence of therapeutic effects for those who do achieve end-of-treatment abstinence. This information will be helpful in determining treatment duration in future clinical trials.

Other secondary outcome measures will include:
1) Daily cigarette consumption assessed by self-reported cigarettes smoked per day.
2) Saliva cotinine concentration, reflecting smoking over the 1-2 days preceding the laboratory assessment.
3) Fagerstrom Test for Nicotine Dependence (FTND) (Heatherton, T. et al., “The Fagerstrom Test for Nicotine Dependence: A Revision of the Fagerstrom Tolerance Questionnaire,” British Journal of Addiction, 1991, 86, 1119-1127).
4) Craving and nicotine withdrawal symptoms, assessed by self-reported ratings on the Minnesota Nicotine Withdrawal Scale (MNWS), widely employed in smoking cessation research Kim, S. et al., “Psychometric Properties of the Minnesota Nicotine Withdrawal Scale: A Korean Version,” Journal of Nursing Measurement, 2007, 15 (2), 121-132. The nine items of the MNWS include the following: urge to smoke (craving); depressed mood; irritability, frustration, or anger; anxiety; difficulty concentrating; restlessness; increased appetite; difficulty going to sleep; and difficulty staying asleep. Each item is rated by a subject on an ordinal scale from 0 (not at all) to 4 (extreme).
5) Self-reported rewarding/aversive effects of cigarettes, using the validated modified Cigarette Evaluation Questionnaire (mCEQ) (Cappelleri, J. et al., “Confirmatory Cigarette Evaluation Questionnaire,” Addictive Behaviors, 2007, 32, 912-923). The mCEQ assesses the degree to which subjects experience the reinforcing effects of smoking, providing five subscale scores: smoking satisfaction (satisfying, tastes good, enjoy smoking), psychological reward (calms down, more awake, less irritable, helps concentrate, reduces hunger), aversion (dizziness, nauseated), enjoyment of respiratory tract sensations (single-item assessment), and craving reduction (single-item assessment).
6) Depression symptoms, including anhedonia, using the CESD-R, a 20-item self-report measure that is widely used in screening for depression.
7) Impulsiveness, assessed by the Recent Rash Impulsivity Scale (RRIS) (Mayhew & Powell, 2014), which is a state (prior 2 weeks) self-report measure that includes factors tapping cognition (planfulness) and behavior (acting rashly without regard to possible negative consequences).
8) The Change in Sexual Functioning Questionnaire is a 14-item self-report measure (different versions for men and women) that assesses (Keller et al., 2006) sexual functioning. The total score as well as five subscales will serve an outcome measure for analysis.
9) Serum amitifadine levels.
10) Nicotine Metabolite Ratio (NMR). This ratio of 3-hydroxycotinine to cotinine is a marker of the metabolic rate for nicotine.
11) Body weight. Amitifadine is predicted to prevent weight gain after quitting smoking.

1. (canceled)

2. A method of treatment for nicotine dependency and nicotine withdrawal effects in a human in need thereof, wherein the method comprises:
a. determining and/or learning if the human is a poor, a slow, an intermediate, a normal, or a fast nicotine metabolizer,
b. determining an effective amount of (1R,5S)-1-(3,4-dichlorophenyl)-3-azabicyclo[3.1.0]hexane, in free or pharmaceutically acceptable salt form, based on whether the human is a poor, a slow, an intermediate, a normal, or a fast nicotine metabolizer, and
c. administering the effective amount of (1R,5S)-1-(3,4-dichlorophenyl)-3-azabicyclo[3.1.0]hexane, in free or pharmaceutically acceptable salt form, to the human.

3. A method of treatment for nicotine dependency and nicotine withdrawal effects in a human in need thereof, wherein the method comprises administering to the human an effective amount of (1R,5S)-1-(3,4-dichlorophenyl)-3-azabicyclo[3.1.0]hexane, in free or pharmaceutically acceptable salt form, and an effective amount of a nicotine replacement therapy, wherein the human is an intermediate, a normal, or a fast nicotine metabolizer.

4-11. (canceled)

12. A method of preventing smoking relapse following quitting smoking in a human in need thereof, wherein the method comprises:
a. determining and/or learning if the human is a poor, a slow, an intermediate, a normal, or a fast nicotine metabolizer,
b. determining an effective amount of (1R,5S)-1-(3,4-dichlorophenyl)-3-azabicyclo[3.1.0]hexane, in free or pharmaceutically acceptable salt form, based on whether the human is a poor, a slow, an intermediate, a normal, or a fast nicotine metabolizer, and
c. administering the effective amount of (1R,5S)-1-(3,4-dichlorophenyl)-3-azabicyclo[3.1.0]hexane, in free or pharmaceutically acceptable salt form.

13-15. (canceled)

16. The method according to claim 2, wherein (1R,5S)-1-(3,4-dichlorophenyl)-3-azabicyclo[3.1.0]hexane is in pharmaceutically acceptable salt form.

17. The method according to claim 16, wherein (1R,5S)-1-(3,4-dichlorophenyl)-3-azabicyclo[3.1.0]hexane in pharmaceutically acceptable salt form is an acid addition salt.

18. The method according to claim 17, wherein (1R,5S)-1-(3,4-dichlorophenyl)-3-azabicyclo[3.1.0]hexane in pharmaceutically acceptable salt form is (1R,5S)-1-(3,4-dichlorophenyl)-3-azabicyclo[3.1.0]hexane hydrochloride.

19. (canceled)

20. The method according to claim 18, wherein (1R,5S)-1-(3,4-dichlorophenyl)-3-azabicyclo[3.1.0]hexane hydrochloride is Polymorph A.

21-27. (canceled)

28. The method according to claim 2, wherein if the human is a poor or slow nicotine metabolizer, then the effective amount of (1R,5S)-1-(3,4-dichlorophenyl)-3-azabicyclo[3.1.0]hexane, in free or pharmaceutically acceptable salt form, is 25 mg to 75 mg per day.

29. The method according to claim 2, wherein if the human is an intermediate, a normal, or a fast nicotine metabolizer, then the effective amount of (1R,5S)-1-(3,4-dichlorophenyl)-3-azabicyclo[3.1.0]hexane, in free or pharmaceutically acceptable salt form, is 100 mg to 200 mg per day.

30. The method according to claim 2, wherein whether the human is a poor, a slow, an intermediate, a normal, or a fast nicotine metabolizer is determined by measuring nicotine metabolite ratio of the human.
